(19) United States
(12) Patent Application Publication
(10) Pub. No.: US 2008/0287478 A1
(43) Pub. Date: Nov. 20, 2008

(54) Nociceptin Analogues and Uses Thereof

(76) Inventors: Lars Bo Laurenborg Hansen, Herlev (DK); Bjarne Due Larsen, Vanlose (DK); Christian Thorkildsen, Roskilde (DK); Carsten Boye Knudsen, Greve (DK)

Correspondence Address: CLARK & ELBING LLP, 101 FEDERAL STREET, BOSTON, MA, 02110, US

(21) Appl. No.: 10/557,869

(22) PCT Filed: May 21, 2004
(86) PCT No.: PCT/DK04/00360

§ 371 Date: May 19, 2008

Related U.S. Application Data
(60) Provisional application No. 60/473,351, filed on May 23, 2003.

(51) Int. Cl. A61K 31/438; C07D 487/10; A61P 29/00; A61P 9/12; A61P 25/20

(52) U.S. Cl. 514/278; 546/18

(57) ABSTRACT

The present invention relates to nociceptin analogues and uses thereof to modulate biological functions. In one aspect, the invention provides modified triazo-spiro compounds that include at least one specialized chemical group that is bound to the compounds. The invention has a wide range of applications including providing a new class of therapeutically useful aquatics.

### FIELD OF THE INVENTION

The present invention relates to nociceptin analogues and uses thereof to modulate biological functions. In one aspect, the invention provides modified triazo-spiro compounds that include at least one specialized chemical group that is bound to the compounds. The invention has a wide range of applications including providing a new class of therapeutically useful aquaretics.

### BACKGROUND

There is almost universal recognition that G-protein coupled receptors are important components of mammalian signalling systems. The receptors are reported to function by helping to convert binding of an extracellular ligand to an internal cell signal. See generally B. Hille (1992) Neuron 9: 187.

It has been disclosed that opioids associate with three classes of G-protein coupled receptors: μ, κ, and δ type. Morphine, enkephalins and benzomorphans are acknowledged ligands of the receptors. See Darland, T et al. (1998) Trends in Neuroscience 21: 215; Simonds, W. F. (1988) Endocr. Rev. 9: 200; and Mollereau, C. et al. FEBS Letters (1994) 341: 33; and references cited therein.

Opioids are believed to influence both the central (CNS) and peripheral (PNS) nervous systems. A wide spectrum of effects are thought to be produced such as analgesia, depression, learning, and memory. Unfortunately, many opioids are associated with unwanted side effects such as dependence and abuse. Accordingly, use of the opioids as pharmacological agents has been limited.

Multiple sub-types of the opioid receptors have been disclosed. One of them is the opioid receptor-like 1 (ORL-1) receptor. The receptor has also been referred to as the orphanin FQ/nociceptin (OFQ/N) receptor. See Henderson, G. et al. (1997); Trends Pharmacol. Sci., (1997) 18: 293; Kapusta, D. R. et al. (1997) Life Sci., 60, PL15; and Darland, T. supra.

A natural ligand of the ORL-1 receptor is believed to be a 17 amino acid peptide called “nociceptin” or “orphanin”. See Darland, T. supra.

Nociceptin reportedly exerts a broad range of CNS and PNS effects. These include modulation of nociception, locomotion, stress and anxiety, food intake, neuroendocrine secretion, learning and memory, and drug addiction. Nociception is thought to be a mechanism in which noxious stimuli are transmitted to the CNS. The ligand is thought to impact smooth muscle tone in the cardiovascular, respiratory, gastrointestinal and urogenital systems. See Meunier, J. C (1997) Eur. J. of Pharm. 340:1; Henderson, G. (1997), supra; and Darland, T. supra.

Certain PNS effects of nociceptin have been reported to include modulation of arterial blood pressure and renal function. The ligand has been reported to be a diuretic with a substantial sodium sparing activity and minimal CNS effect at particular doses. See Kapusta, D. R. et al. (1997), supra.

However there is growing recognition that nociceptin may not be a suitable pharmacological agent in all settings. For example, it has poor oral availability and may be subject to degradation in vivo. Accordingly, there have been efforts to identify small molecules with ORL-1 receptor activity. See Jenck, F. et al. (1997) PNAS (USA) 97: 4938; and Dautzenberg, F. M et al. (2001) J. of Pharm. And Experimental Ther. 298: 812.

For instance, there have been efforts to make and use certain triaza-spiro compounds as nociceptin replacements. The compounds have been reported to interact with the ORL-1 receptor and may be useful to treat some diseases. See U.S. Pat. Nos. 6,075,034; 6,071,925; 6,277,991; WO 99/59997; and EP 0 921 125 A1.

Certain 1-phenyl-1,3,8-triazaspiro[4,5]decan-4-ones have also been disclosed. See U.S. Pat. Nos. 3,238,216; and 3,161,644.

ORL-1 receptor agonists have been reported such as certain 4-(2-keto-1-benzimidazolonyl)piperidines and azacyclic compounds. See WO 99/36421 and WO 01/07050.

Nociceptin has been reported to be an “aquaretic” ie., a diuretic with substantial sodium and/or potassium ion sparing activity. See Kapusta, D. R. et al. (1997), supra; Kapusta, D. R. (2001), supra.

Other diuretic compounds are known and are generally useful to assist loss of undesired water from the body. Unfortunately, many diuretics also cause an unwanted loss of urinary sodium and potassium. Loss of these ions can impact a wide range of medical disorders including edema associated with hyponatremia.

Many compounds that reportedly interact with the ORL-1 receptor are believed to have substantial drawbacks. For example, it has been difficult to make compounds that selectively modulate the receptor and avoid unwanted CNS effects. Additionally, there have been few successful attempts to make compounds that avoid nervous system effects but still modulate diuresis.

It would be useful to have compounds that interact with the ORL-1-receptor and modulate diuresis. It would be especially useful to have aquaretic compounds that are orally available and exhibit minimal CNS effects.

### SUMMARY OF THE INVENTION

The present invention relates to nociceptin analogues that can be used to modulate a variety of biological functions. In one aspect, the invention provides a modified triaza-spiro compound that includes at least one elongated chemical group. Particular invention compounds feature reduced impact on the central nervous system (CNS) and, in some instances, better oral availability. Practice of the invention has a range of important applications including providing modified triaza-spiro compounds that function as therapeutically useful aquaretics.

It has been found that by modifying certain triazo-spiro molecules it is possible to provide them with a range of desirable biological functions. More specifically, it has been found that by adding at least one elongated chemical group such as what is referred to herein as a “polar tail group”, it is possible to reduce or in some cases avoid unwanted penetration of the triazo-spiro molecules into the CNS. In some instances, it is also possible to increase oral bioavailability and enhance peripheral nervous system (PNS) activity of the compounds. More particular invention compounds are modified 1,3,8-triaza-spiro[4.5]decan-4-ones that include the polar tail group covalently attached thereto. In most cases the polar tail group is covalently linked to the 3-position of the 1,3,8-triaza-spiro[4.5]decan-4-one, although other linking sites such as to an optionally substituted moiety or group (e.g., an aromatic ring) may be more suitable for other applications.

More specific polar tail groups in accord with the invention are substantially charged chemical moieties especially at about physiological pH. Preferred polar tail groups are elongated and serve a linker function that is intended to space the charge away from the core 1,3,8-triaza-spiro[4.5]decan-4-one. In some invention embodiments, the linker contributes to at least some of the charge of the polar tail group.

Particular elongated chemical groups serve a linker function in which the linker part is substantially apolar including, in some instances, being significantly hydrophobic. In this example, the elongated chemical group typically spaces multiple 1,3,8-triaza-spiro[4.5]decan-4-one molecules from each other, usually two of such molecules. Examples of suitable elongated chemical groups including polar tail groups are provided in Formulae I-III as shown below.

These features of the invention provide important advantages.

For instance, the linker function of the elongated chemical group, and particularly the polar tail group, can be rigid or flexible as needed to suit an intended application. Standard synthetic manipulations can be used to modify the group so as to position charge near the core 1,3,8-triaza-spiro[4.5]decan-4-one or relatively far away from it. The concept of spacing charge away from the core molecule has been found to modulate the activity of the 1,3,8-triaza-spiro[4.5]decan-4-one, for instance, by providing at least one of increased bioavailability, reduced penetration into the CNS, increased ORL1 receptor binding, and enhanced PNS activity. As is discussed below, particular polar tail groups of interest are especially useful in reducing penetration of the 1,3,8-triaza-spiro[4.5]decan-4-one molecule into the CNS. Certain of such molecules have been found to be therapeutically useful aquaretics as discussed below.

Accordingly, and in one aspect, the invention provides a 1,3,8-triaza-spiro[4.5]decan-4-one compound represented by the following formula I:

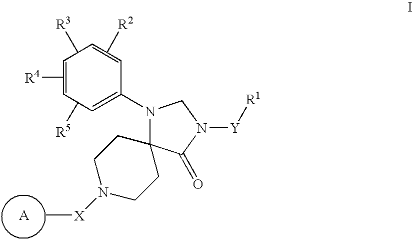

wherein,

(a) Y is 0, an optionally substituted C₁₋₁₂ alkylene, C₁₋₁₂ alkenylene, C₁₋₁₂ alkynylene group, 2-6 peptidyl residue or poly oxyalkyl or combinations thereof in which each alkyl, alkenyl or alkynyl group is branched or unbranched,

(b) R¹ is —NR⁶, R⁷, R⁸ in which each of R⁶ and R⁷ is independently H or optionally substituted lower alkyl or R⁶ is —(CH₂)ₙ₁—NHR₇ in which n1 is between from about 1 to about 20 and R⁸ is 0, H or optionally substituted lower alkyl,

(c) R¹ is NR³—[(CH₂)ₙ₂—NH]ₙ₃—(CH₂)ₙ₄—R⁹ in which n2 and n3 are each independently 1 to about 10, n4 is 1 to about 6, R⁹ is —NR⁶, R⁷, cyano, or an optionally substituted hydrazine, guanidine, azole or azine group,

(d) R¹ is —NH—[(CH₂)ₙ₁—NH]ₙ₂—(CH₂)ₙ₃—X2 in which each of R¹⁰ and R¹¹ is independently —NR⁶, R⁷, —CH═NH, cyano, or 0, provided that both of R¹⁰ and R¹¹ are not 0, wherein X2 is represented by the following formula

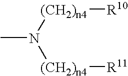

or

(e) R¹ is represented by the following group:

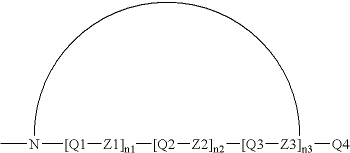

in which each of Q1, Q2, Q3 and Q4 are independently an optionally substituted lower alkyl, lower oxyalkyl, α,ω-dioxo-lower alkyl, or aryl alkyl group, and each of Z1, Z2 and Z3 is independently N, O or S,

(f) R¹ is an optionally substituted lower alkoxy, lower alkylcarboxy group, allyl, halogen, benzoxy, or a Boc protecting group,

(g) A is an optionally substituted C₅₋₁₂ cycloalkyl (e.g., cyclohexyl), phenyl, aminophenyl, cyanophenyl, cyanodiphenylmethyl, phenoxy, benzodioxinyl, cyanodiphenylmethyl, napthyl, anthryl, furanyl, indanyl, azulenyl, indolyl, isoindolyl, benzothienyl, benzofuranyl, bicyclo[6.2.0]dec-9-yl, acenapthenyl, bicyclo[3.3.1]non-9-yl, phenalenyl, indenyl, bicyclo[3.1.0]hex-3-yl, or coumarinyl group,

(h) X is a 0, or an optionally substituted lower alkyl, lower alkenyl, or lower alkynyl group,

(i) R², R³, R⁴, and R⁵ are each independently H, halogen or an optionally substituted lower alkyl;

and a salt or a solvate thereof, preferably a pharmaceutically acceptable salt or solvate.

In the foregoing representation of the compounds of Formula I, the elongated chemical group is represented by —Y—R¹. That group is referred to as a polar tail group in embodiments in which the elongated chemical group includes a least one moiety that is charged at about physiological pH (e.g., amine, amino, carboxy, and the like). In a preferred embodiment of the compound represented by Formula I, if A comprises a phenyl group annulated or as a substituent, then R¹ comprises more than one amino or guanidino group.

In another aspect, the present invention provides a method of malting the compound represented by Formula I as shown above. In one embodiment, the method includes at least one and preferably all of the following steps:

a) alkylating the 3-position of an triaza-spiro compound such as an optionally substituted 1,3,8-triaza-spiro[4.5]decan-4-one,

b) aminating the product of step a) under reducing conditions sufficient to add the A ring to the 8-position of the product,

c) brominating the alkyl group added to the 3-position of the product of Step b) to produce a bromide; and

d) substituting the bromine with the R¹ group to make the compound.

However in another invention aspect, compounds are provided in which two triazo-spiro molecules are covalently linked together by a polar tail group or another elongated chemical group that can be substantially apolar or in some cases relatively hydrophobic. In either case, the function of the linking group is to space the triazo-spiro compounds from each other and in some embodiments to distribute charge or hydrophobicity therebetween. Particular linking groups of interest help reduce or eliminate CNS penetration as determined by tests disclosed herein.

In one embodiment, such compounds are represented by the following formula III:

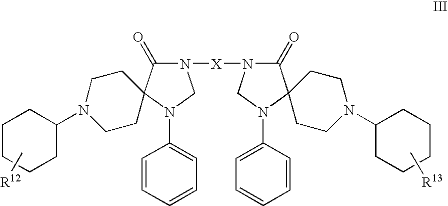

wherein,
(a) R¹² and R¹³ are each independently an optionally substituted lower alkyl or lower alkoxy group,
(b) X is an elongated chemical group, preferably an optionally substituted lower alkyl group, 2-6 peptidyl residue or a polymer; and a salt or solvate thereof, preferably a pharmaceutically acceptable salt thereof.

As discussed below, particular compounds of the invention are orally available and peripherally acting nociceptin receptor (ORL-1) agonists. More specific compounds feature substantial binding and efficacy towards the ORL1 receptor as detected by assays disclosed herein. Additional compounds of interest feature potassium- and sodium-sparing aquaretic activity, also as determined by assays described in more detail below.

In another aspect, the invention provides a composition, preferably one that is pharmaceutically acceptable, that includes at least one, preferably less then ten, and more preferably one, two, three, or four of the compounds disclosed herein. Particular compositions of interest are pharmaceutically acceptable and include at least one acceptable carrier or vehicle.

The invention also provides a method of modulating diuresis in a mammal. In one embodiment, the method includes administering to the mammal at least one composition of the invention (preferably less then five, more preferably one or two of same) in an amount sufficient to modulate the diuresis in the mammal.

Further provided is a method of modulating aquaresis in a mammal that in one embodiment includes administering to the mammal at least one composition of the invention (preferably less then five, more preferably one or two of same) in an amount sufficient to modulate the aquaresis in the mammal.

The present disclosure also provides a method for preventing or treating edema in a mammal. In one example, the method includes administering to the mammal at least one composition of the invention (preferably less then five, more preferably one or two of same) in an amount sufficient to prevent or treat the edema such as pulmonary edema or edema associated with hyponatremia.

Also disclosed is a method of modulating arterial blood pressure in a mammal. In one embodiment, the method includes administering to the mammal at least one composition of the invention (preferably less then five, more preferably one or two of same) in an amount sufficient to modulate the arterial blood pressure in the mammal.

Further provided by the present invention is a method of antagonizing the nociceptin (ORL1) receptor. In one embodiment, the method includes contacting the receptor with an effective amount of at least one of the compounds or compositions disclosed herein, preferably less then five, more preferably one or two of same).

### BRIEF DESCRIPTION OF THE DRAWINGS

FIG. 1 is a schematic drawing showing a preferred method of making a triazo-spiro compound that includes a polar tail group.

FIGS. 2A-D show the compound number, structure, and selected characteristics of several 3-substituted 8-(4-isopropyl-cyclohexyl)-1-phenyl-1,3,8-triaza-spiro[4.5]decan-4-ones made as described in the Examples.

### DETAILED DESCRIPTION OF THE INVENTION

As discussed, the invention generally relates to nociceptin analogues and uses thereof to modulate one or a combination of different biological functions. In one aspect, the invention provides triazo-spiro compounds comprising at least one polar tail group, preferably about one or two of such polar tail groups. The invention has a wide range of applications including use as therapeutically useful aquaretics.

In one embodiment, the compound generally represented as Formula I above, can be more particularly represented by the formula II:

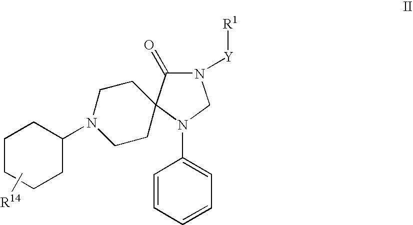

in which Y is 0, R¹⁴ is halogen, cyano, hydroxy, nitro, or an optionally substituted lower alkyl, lower alkenyl, or lower alkynyl group and R¹ is the same as defined previously for Formula I.

Specifically excluded from the compounds represented by Formulae I and II as shown above are compounds disclosed in WO 99/59997 (PCT/DK/00266) including 3-(7-Aminoheptyl)-8-naphthalen-1-ylmethyl-1-phenyl-1,3,8-triaza-spiro[4.5]decan-4-one; 3-(5-aminopentyl)-8-naphthalen-1-ylmethyl-1-phenyl-1,3,8-triaza-spiro[4.5]decan-4-one; 3-(9-aminononyl)-8-napthalen-1-ylmethyl-1-phenyl-1,3,8-triaza-spiro[4.5]decan-4-one; 3-(3-dimethylaminopropyl)-8-napthalen-1-ylmethyl-1-phenyl-1,3,8-triaza-spiro[4.5]decan-4-one; N-(5-(8-Naphthalen-1-ylmethyl-4-oxo-1-phenyl-1,3,8-triazaspiro[4.5]dec-3-yl)pentyl)guanidine; N-(2-aminoethyl)-2-(8-naphthalen-1-ylmethyl-4-oxo-1-phenyl-1,3,8-triazaspiro[4.5]dec-3-yl)acetamide; N-(3-aminopropyl)-2-(8-naphthalen-1-ylmethyl-4-oxo-1-phenyl-1,3,8-triazaspiro[4.5]dec-3-yl)acetamide; N-(2-guanidinoethyl)-2-(8-naphthalen-1-ylmethyl-4-oxo-1-phenyl-1,3,8-triazaspiro[4.5]dec-3-yl)acetamide; as well as specified salts thereof.

By the phrase “optionally substituted” is meant substitution by other than hydrogen at one or more available positions, typically 1 to 3 or 4 positions, by one or more suitable groups such as those disclosed herein.

Suitable groups that may be present on a “substituted” group, moiety or other site as disclosed herein include halogen such as fluoro, chloro, bromo and iodo; cyano; hydroxyl; nitro; azido; alkanoyl such as a C₁₋₆ alkanoyl group such as acyl and the like; carboxamido; lower alkyl; lower alkenyl; lower alkynyl; lower alkoxy, aryloxy such as phenoxy; alkylthio groups including those moieties having one or more thioether linkages and from 1 to about 12 carbon atoms, or 1, 2, 3, 4, 5 or 6 carbon atoms; alkylsulfinyl groups including those moieties having one or more sulfinyl linkages and from 1 to about 12 carbon atoms, or 1, 2, 3, 4, 5, or 6 carbon atoms; alkylsulfonyl groups including those moieties having one or more sulfonyl linkages and from 1 to about 12 carbon atoms, or 1, 2, 3, 4, 5, or 6 carbon atoms; aminoalkyl groups such as groups having one or more N atoms and from 1 to about 12 carbon atoms, or 1, 2, 3, 4, 5 or 6 carbon atoms; carbocyclic aryl having 6 or more carbons, particularly phenyl (e.g., an R group being a substituted or unsubstituted biphenyl moiety); aralkyl having 1 to 3 separate or fused rings and from 6 to about 18 carbon ring atoms such as benzyl; aralkoxy having 1 to 3 separate or fused rings and from 6 to about 18 carbon ring atoms, such as O-benzyl; or a heteroaromatic or heteroalicyclic group having 1 to 3 separate or fused rings with 3 to about 8 members per ring and one or more N, O or S atoms, e.g., coumarinyl, quinolinyl, pyridyl, pyrazinyl, pyrimidyl, furyl, pyrrolyl, thienyl, thiazolyl, oxazolyl, imidazolyl, indolyl, benzofuranyl, benzothiazolyl, tetrahydrofuranyl, tetrahydropyranyl, piperidinyl, morpholino and pyrrolidinyl.

As used herein, the term “lower alkyl” denotes a straight- or branched-chain alkyl group containing from 1 to 8 carbon atoms, for example, methyl, ethyl, propyl, isopropyl, n-butyl, i-butyl, 2-butyl, t-butyl, and the like. An acceptable lower alkyl group is typically positioned cis to the nitrogen atom of the azine ring. A trans configuration may be more appropriate for some invention applications.

By “lower alkenyl” is meant a straight- or branched-chain alkyl group containing from 1 to 8 carbon atoms that includes a least one C═C bond such as ethylene, propylene, isopropylene, n-butylene, and the like.

The term “lower alkynyl” as used herein denotes a straight- or branched-chain alkyl group containing from 1 to 8 carbon atoms that includes a least one carbon-carbon triple bond such as ethynyl, propynyl, and the like.

The term “lower alkoxy” denotes a group wherein the alkyl residues is as defined above, and which is attached via an oxygen atom.

The term “salt” and particularly “pharmaceutically acceptable salt” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methane-sulfonic acid, p-toluenesulfonic acid and the like.

In the foregoing representation of the invention compound shown by Formula II, R¹² is preferably a lower alkyl group such as an optionally substituted n-propyl or isopropyl group. More preferably, the R¹² group is unsubstituted and is bound to the 4-position of the cyclohexyl ring.

As mentioned, compounds of the invention are more generally represented by Formula I as shown above. In one embodiment, R¹ includes at least one primary amine group, preferably one, two or three of same. Alternatively, R¹ includes at least one secondary amine group, preferably one, two or three of such groups. In another embodiment, R¹ includes a tertiary amine group or a polyamine.

In another example of the compound generally represented above as Formula I, R¹ includes a cyclic amine group such as that group shown in part (e) of Formula I. In this invention embodiment, each of Q1, Q2, Q3 and Q4 as shown in part (e) of Formula I above can be lower alkyl such as ethyl, propyl, butyl, pentyl, or hexyl. More specific examples of suitable lower alkyl groups are represented below as optionally substituted formulae:

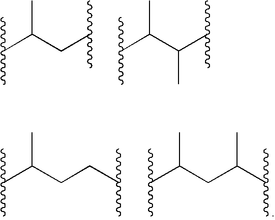

In another embodiment, Q1, Q2, Q3 and Q4 as shown in part (e) of Formula I above can be an optionally substituted lower alkoxy such as shown by the following formulae:

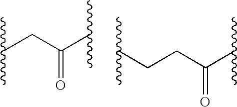

In still another embodiment, Q1, Q2, Q3 and Q4 as shown in part (e) of Formula I above can be an optionally substituted α, or ω-dioxo-lower alkyl, as represented by the following formulae:

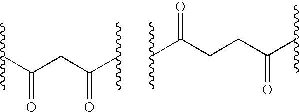

In another invention embodiment, Q1, Q2, Q3 and Q4 as shown in part (e) of Formula I above can be an optionally substituted aralkyl as represented by the following formulae:

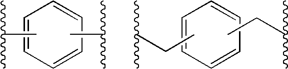

Particular examples of a cyclic amines in accord with the invention are represented below in the following formulae showing (from left to right) a cyclam, 5,7-dioxoxcyclam, and 5,12-dioxocyclam:

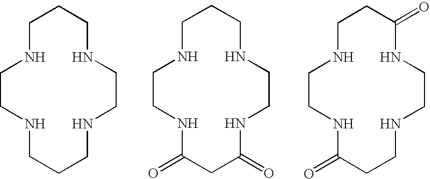

As mentioned, it is an object of the present invention to provide a wide range of modified 1,3,8-triaza-spiro[4.5]decan-4-ones that include at least one polar tail group covalently attached thereto such as in the 3-position. Preferably, an elongated chemical group such as the polar tail group has a molecular weight of less than about 1000 Da as determined by routine sizing techniques. More specifically, the R¹ group as shown above in Formula I will have molecular weight of less than about 1000 Da. A more particular R¹ group has a net positive charge of between 1 to about 10 at a pH of about 7.5 as determined by standard approaches such as inspection of chemical group ionization (pKa, pKb) tables.

It is also an object of this invention to provide modified 1,3,8-triaza-spiro[4.5]decan-4-ones that can be administered to a mammal by one or a combination of routes which generally include injection such as intravenously (i.v.) intracerebroventricularly (i.c.v), intraplantarally (i.pl.), intraperitoneally (i.p.), intrathecally (i.t.); or per oral administration (p.o.). Other potential administration routes are discussed below including nasal, vaginal and suppository use as well as depot routes.

In embodiments in which a compound of the invention is intended to be orally available (bioavailability), it will be generally preferred to have compounds that exhibit an oral bioavailability (F %) of at least about 1%, preferably at least about 10%, more preferably at least about 20% or 30%, even more preferably up to about 50%, such as up to 75% as determined by a standard plasma test.

By the term “standard plasma test” or like phrase is meant an assay described by the following general steps:
a) administering at least one of the compounds disclosed herein to a mammal such as a rabbit, rodent or like experimental animal, preferably one or two of such compounds,
b) collecting blood samples over multiple time intervals and preparing plasma therefrom; and
c) detecting and optionally quantifying the compound in the plasma by one or a combination of standard methods such as chromatography such as liquid chromatography (LC) optionally coupled to a suitable detector such as a mass spectrometer or analyzer.

See also Milo Gibal (1991) Biopharmaceutics and Pharmacology, 4th edition (Lea and Sediger). Peptides with good oral availability are those which are observed in plasma generally within about 30-60 minutes after oral administration.

A preferred detection system for use with the standard plasma test is an LC/MS/MS system as described below in the Examples. Typically, one or two compounds are administered to the test animal eg., i.v. or p.o. bolus and blood collected between from about 0 to 600 minutes, preferably 0 to about 300 minutes. Methods for making plasma from blood are standard in the field. Sometimes, use of an appropriate control will be desirable including mock injection of compound e.g., by use of water, buffer, etc. If desired, standard compound curves can be used to quantify the amount of compound in a sample.

Additionally preferred compounds of the present invention exhibit an increase in diuresis of at least 1.2 as determined by a standard diuresis test. In one embodiment, that increase in diuresis is between 1.5 to about 5.0 as determined in the standard diuresis test, such as between 1.5 to about 4.5, for example between 2.0 to about 4.0, such as between 2.5 to about 3.5.

As will be appreciated, diuresis can be readily measured by one or a combination of standard laboratory approaches. Methods for characterizing a variety of diuretics have been described. See E. K. Jackson in Goodman & Gilman's The Pharmacological Basis of Therapeutics 9^{th} Ed. (Chapters 29-31, pp. 685-758) McGraw-Hill, New York, (NY).

Diuretics are defined herein as a class of therapeutic agents that help adjust body fluid volume and or composition. Such diuretics (as well as particular aquaretics of the invention) can be used to prevent, treat, or reduce the severity of a wide spectrum of medical conditions such as hypertension, acute or chronic heart failure, acute or chronic renal failure, nephrotic syndrome, and cirrhosis. More particular examples of diuretics include, but are not limited to, carbonic anhydrase inhibitors, osmotic diuretics, high ceiling diuretics, loop diuretics, thiazide and thiazide-like diuretics, potassium-sparing diuretics, aldosterone antagonists, vasopressin and other agents. See E. K. Jackson, supra, for more information about diuretics and use thereof.

In one approach referred to herein as a “standard diuresis test” or related phrase, the following general steps are preformed.
a) administering at least one of the compounds disclosed herein to a mammal such as a rabbit, rodent or like experimental animal, preferably one or two of such compounds,
b) collecting urine from the mammal over multiple time intervals, preferably less then about 24 hours, more preferably between from about 1 to about 10 hours; and
c) measuring the amount (volume) of urine collected from the mammal.

Sometimes, use of an appropriate control will be desirable including mock injection of compound e.g., by use of water, buffer, etc. If desired, standard compound curves can be used to quantify the amount of compound in a sample. See the Example 35 below for a particular illustration of the standard diuresis test.

Additionally preferred compounds of the invention provide good inhibitory activity in what is referred to herein as a standard hORL1 receptor binding assay. Preferably, the compound exhibits an IC₅₀ of at least about 0.1 nM in the assay, more typically between from about 1 nM to about 100 nM in the assay, such as between 2 nM to about 90 nM, for example between 5 nM to about 80 nM, such as between 10 nM to about 70 nM, for example between 15 nM to about 60 nM, such as between 20 nM to about 50 nM, for example between 25 nM to about 45 nM, such as between 30 nM to about 40 nM.

Reference herein to “standard hORL1 receptor binding assay” or like phrase means performing the following general steps.
a) making a human orphanin receptor (hORL1) preparation in a suitable binding buffer,
b) contacting the preparation with at least one of the invention compounds, preferably one or two of same, and also contacting with detectably-labelled nociceptin (e.g., tritium-labelled) either alone or with cold (unlabeled) nociceptin; and
c) detecting the amount of binding between the detectably-labelled nociceptin and the hORL1 receptor as being indicative of the inhibitory activity of the compound.

See Example 36 below, disclosing a particular illustration of the standard hORL1 receptor binding assay. See also U.S. Pat. No. 6,071,925 (disclosing another suitable ORL1 (“OFQ”) binding assay.

Further compounds in accord with the invention provide acceptable activity in what is referred to herein as a standard forskoline-induced cAMP assay. In one embodiment, the compound exhibits an EC₅₀ of less than about 50 nM in a standard forskoline-induced cAMP assay, such as less than 40 nM, for example less than 30 nM, such as less than 20 nM, for example less than 10 nM.

A generally preferred standard forskoline-induced cAMP formation assay is generally described as follows:
a) contacting cells in a physiologically acceptable buffer such as D-PBS with at least one and preferably all of the following components:
   i) between from about 0.5 mM to about 5 nM, preferably about 2 mM of a suitable phosphodiesterase blocker such as IBMX,
   ii) between from about 1 micromolar to about 50 micromolar forskoline (stimulates cAMP formation), preferably about 10 micromolar,
   iii) at least one invention compound, preferably one, having a concentration of between from about 0.01 nM to about 100 nM, preferably 0.1 nM to about 10 nM, more preferably about 0.6 nM
b) incubating the cells at 37° C. for less than about 1 hour, preferably between from about 5 minutes to about 30 minutes, to produce cAMP,
c) increasing the pH of the buffer sufficient to produce an extract from the cells eg., by adding a concentrated acid such as HCL; and
d) measuring the cAMP produced as being indicative of the ability of the test compound to modulate (increase or decrease) cAMP production by the cells.

Particularly preferred invention compounds can be shown to inhibit forskoline-stimulated cAMP formation in the assay. The standard forskoline-induced cAMP formation assay can be used with a variety of suitable controls. In one example of the assay, the compound is substituted with a mock sample (water, saline, buffer etc.). A particular example of the assay is shown below in Example 37.

More specific compounds of the invention as shown in Formula I include the following:
(a) cis-3-(6-Methylamino-hexyl)-8-(4-isopropyl-cyclohexyl)-1-phenyl-1,3,8-triaza-spiro[4.5]decan-4-one (Compound 12),
(b) trans-3-(6-Methylamino-hexyl-8-(4-isopropyl-cyclohexyl)-1-phenyl-1,3,8-triaza-spiro[4.5]decan-4-one (Compound 13),
(c) cis-3-N-(6-Methylaminohexyl)-(6-methylaminohexyl)-8-(4-isopropyl-cyclohexyl)-1-phenyl-1,3,8-triaza-spiro[4.5]decan-4-one (Compound 14),
(d) trans-3-N-(6-Methylaminohexyl)-(6-methylaminohexyl)-8-(4-isopropyl-cyclohexyl)-1-phenyl-1,3,8-triaza-spiro[4.5]decan-4-one (Compound 15),
(e) cis-3-(3-Amino-propyl)-8-(4-isopropyl-cyclohexyl)-1-phenyl-1,3,8-triaza-spiro[4.5]decan-4-one (Compound 16),
(f) trans-3-(3-Amino-propyl)-8-(4-isopropyl-cyclohexyl)-1-phenyl-1,3,8-triaza-spiro[4.5]decan-4-one (Compound 21),
(g) cis-3-(9-Amino-nonyl)-8-(4-isopropyl-cyclohexyl)-1-phenyl-1,3,8-triaza-spiro[4.5]decan-4-one (Compound 23),
(h) trans-3-(9-Amino-nonyl)-8-(4-isopropyl-cyclohexyl)-1-phenyl-1,3,8-triaza-spiro[4.5]decan-4-one (Compound 24),
(i) cis-3-(13-Aminoethyl-10,13,16-triazahexadecan)-8-(4-isopropyl-cyclohexyl)-1-phenyl-1,3,8-triaza-spiro[4.5]decan-4-one (Compound 31),
(j) trans-3-(13-Aminoethyl-10,13,16-triazahexadecan)-8-(4-isopropyl-cyclohexyl)-1-phenyl-1,3,8-triaza-spiro[4.5]decan-4-one (Compound 32),
(k) cis-3-(3-Dimethylamino-propyl)-8-(4-isopropyl-cyclohexyl)-1-phenyl-1,3,8-triaza-spiro[4.5]decan-4-one (Compound 26),
(l) cis-3-(6-Amino-hexyl)-8-(4-isopropyl-cyclohexyl)-1-phenyl-1,3,8-triaza-spiro[4.5]decan-4-one (Compound 22),
(m) cis-3-(9-Dimethylamino-nonyl)-8-(4-isopropyl-cyclohexyl)-1-phenyl-1,3,8-triaza-spiro[4.5]decan-4-one (Compound 28),
(n) cis-3-(7-Aminoethyl-4,7,10-triazadecan)-8-(4-isopropyl-cyclohexyl)-1-phenyl-1,3,8-triaza-spiro[4.5]decan-4-one (Compound 29),
(o) cis-3-(10-Aminoethyl-7,10,13-triazamidecan)-8-(4-isopropyl-cyclohexyl)-1-phenyl-1,3,8-triaza-spiro[4.5]decan-4-one (Compound 30),
(p) cis-3-(6-Dimethylamino-hexyl)-8-(4-isopropyl-cyclohexyl)-1-phenyl-1,3,8-triaza-spiro[4.5]decan-4-one (Compound 27),
(q) cis-8-(4-Isopropyl-cyclohexyl)-3-(10,14,17,20,23-pentaazatricosanyl)-1-phenyl-1,3,8-triaza-spiro[4.5]decan-4-one (Compound 35),
(r) cis-8-(4-Isopropyl-cyclohexyl)-1-phenyl-3-[9-(1,4,8,11-tetraaza-cyclotetradec-1-yl)-nonyl]-1,3,8-triaza-spiro[4.5]decan-4-one (Compound 39),
(s) cis-8-(4-Isopropyl-cyclohexyl)-3-(7,10,14,17,20-pentaazaeicosanyl)-1-phenyl-1,3,8-triaza-spiro[4.5]decan-4-one (Compound 34),
(t) cis-8-(4-Isopropyl-cyclohexyl)-3-(4,7,10,14,17-pentaazaheptadecyl)-1-phenyl-1,3,8-triaza-spiro[4.5]decan-4-one (Compound 33),
(u) cis-8-(4-Isopropyl-cyclohexyl)-1-phenyl-3-[3-(1,4,8,11-tetraaza-cyclotetradec-1-yl)-propyl]-1,3,8-triaza-spiro[4.5]decan-4-one (Compound 36); and
(v) cis-8-(4-Isopropyl-cyclohexyl)-1-phenyl-3-[6-(1,4,8,11-tetraaza-cyclotetradec-1-yl)-hexyl]-1,3,8-triaza-spiro[4.5]decan-4-one (Compound 38), including a salt or solvate thereof, preferably a pharmaceutically acceptable salt.

In embodiments in which it is desirable to reduce activity of an invention compound within the CNS, such a compound will suitably exhibit negligible penetration into the CNS. Methods for detecting penetration through the blood-brain barrier (BBB) are known and include recognized tests as reported by Jenck et al.; PNAS (2000) 97 (9), 4938, for instance.

As discussed, the invention also provides compounds according to Formula III in which the elongated chemical group is linked to two core triazo-spiro molecules. In one embodiment, the lower alkyl and lower alkoxy groups featured in part (a) of Formula III are each independently substituted with at least one of halogen, cyano, hydroxy or nitro. Alternatively, or in addition, the lower alkyl group is substituted with between from 1 to about 5 nitrogen atoms. In another embodiment, each of R¹² and R¹³ as shown in Formula III above is an unsubstituted lower alkyl group the same or different such as n-propyl or isopropyl. Such groups can be covalently linked to the compound in a variety of suitable ways including by linking to the cyclohexyl group at the 4-position.

Referring now to Formula III as shown previously, the linking chemical group (here defined as X) can be an optionally substituted lower alkyl or other alkyl group such as heptyl, octyl, nonyl, or decyl group. Other suitable polar tail groups 5-azaundecan, 6-azamidecan, 7-azapentadecanl, 8-azaheptadecan, 9-aza-nonadecan, 10-azaundodecan, 5-azaundecan-1,11-diyl, 6-azamidecan 1,13-diyl, 7-azapentadecanl-1,15-diyl, 8-azaheptadecan 1,17-diyl, 9-aza-nonadecan-1,19-diyl or a 10-azaundodecan-1,2′-diyl group.

Examples of suitable linking groups X include 2-6 peptidyl residue, polymers having a molecular weight of between from about 100 to about 700, preferably about 150 such as polystyrene, polyethylene glycol (PEG), polyamine. Other acceptable linkers include particular disulfides such as cystine, homocystine, and their N-protected analogues preferably acylated to the 3-position. Suitable peptidyl residue linking groups include homo- or heteropolymers of one or more of the 20 common amino acids eg., (Ala)₂₋₆, (Leu)₂₋₆, (Ala-Isoleu)₂₋₆, and the like.

In one embodiment, an acceptable linker that includes the disulfide is represented by the following Formula IIIA:

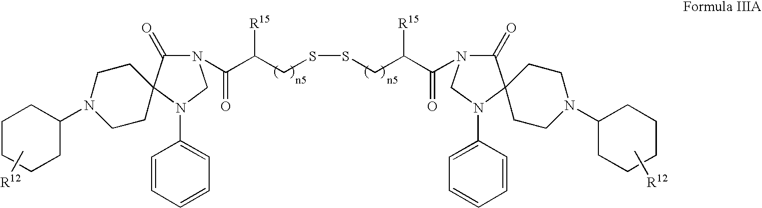

In which R¹⁵ is 0 (null) or an optionally substituted amino group such as an aacetamido group, n5 is about 1 to about 3; and R¹² is an optionally substituted lower alkyl or lower alkoxy group.

Preferred molecules according to Formula IIIA above, are essentially symetrical and are structrually different from the molecules represented by Formula III. Methods of making the molecules shown in Figure IIIA are straightforward and include reacting commercially available preparations of cystine and homocystine along lines of methods disclosed herein. Also contemplated are protected derivatives of the molecules shown in Figure IIIA including, but not limited to, embodiments in which R¹⁵ is NH-Boc, NH-Ac or NH-Fmoc.

It will be apparent from the foregoing discussion that the elongated chemical group shown in Formula III can be substantially polar, apolar or hydrophobic as needed to produce the desired compound.

More particular compounds of Formula III have at least one of the following properties: an increase in diuresis of at least 1.2 as determined by the standard diuresis test, preferably in which the increase in diuresis is between 1.5 to about 5.0 as determined in the standard diuresis test; b) an IC₅₀ of at least about 1 nM in a standard hORL-1 receptor binding assay, preferably an IC₅₀ of between from about 5 nM to about 100 nM in the standard hORL-1 receptor binding assay, c) an EC₅₀ of less than about 50 nM in a standard forskoline-induced cAMP assay.

Examples of more particular invention compounds according to Formula III as shown above include the following.
(i) bis-(cis-3-Propyl-8-(4-isopropyl-cyclohexyl)-1-phenyl-1,3,8-triaza-spiro[4.5]decan-4-on)-amine (Compound 19),
(ii) bis-(trans-3-Propyl-8-(4-isopropyl-cyclohexyl)-1-phenyl-1,3,8-triaza-spiro[4.5]decan-4-on)-amine (Compound 20), and
(iii) 1,9-bis-(cis-8-(4-isopropyl-cyclohexyl)-1-phenyl-1,3,8-triaza-spiro[4.5]decan-4-one-3-yl)-nonane (Compound 25).

The invention compounds of formula I, II and III as well as corresponding racemates, enantiomers, salts, solvates, and pharmaceutically acceptable salts thereof can be used as therapeutically useful compositions (medicaments). For instance, in the form of pharmaceutical preparations. The pharmaceutical preparations can be administered orally, e.g. in the form of tablets, coated tablets, dragees, hard and soft gelatin capsules, solutions, emulsions or suspensions. The administration can, however, also be effected rectally, e.g. in the form of suppositories, or parenterally, e.g. in the form of injection solutions. Other injection routes have already been mentioned including (i.v.) intracerebroventricularly (i.c.v), intraplantarally (i.pl.), intraperitoneally (i.p.), and intrathecally (i.t.) administration. In embodiments in which an injection route is favored, bolus administration may be helpful in many instances.

The invention compounds of formula I, II and III as well as corresponding racemates, enantiomers, salts, solvates, and pharmaceutically acceptable salts thereof can be processed with pharmaceutically inert, inorganic or organic excipients for the production of tablets, coated tablets, dragees and hard gelatin capsules. Lactose, corn starch or derivatives thereof, talc, stearic acid or its salts etc. can be used as such excipients e.g. for tablets, dragees and hard gelatin capsules. Suitable excipients for soft gelatin capsules are e.g. vegetable oils, waxes, fats, semi-solid and liquid polyols etc. Suitable excipients for the manufacture of solutions and syrups are e.g. water, polyols, saccharose, invert sugar, glucose etc. Suitable excipients for injection solutions are e.g. water, alcohols, polyols, glycerol, vegetable oils etc. Suitable excipients for suppositories are e.g. natural or hardened oils, waxes, fats, semi-liquid or liquid polyols etc. Moreover, the pharmaceutical preparations can contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

For some invention applications, administration via a depot formulation may be highly desirable. See eg. U.S. Pat. Nos. 5,407,609 and 5,654,008 for additional information. Liposomes, microsphere and liquid stabilizer based formulations are also within the scope of the present invention.

Compositions according to the invention typically include at least one of the compounds disclosed herein, preferably less than five of same, more preferably one or two of such compounds, with at least one pharmaceutically acceptable carrier or vehicle. Such compositions can be used as the sole active agent or in combination with other agent(s) such as in approaches using a “cocktail” format ie., a combination of different active agents.

For example, in embodiments in which one or more of the compositions or compounds disclosed herein is used as a diuretic, such a formulation can be employed alone or in combination with at least one recognized diuretic agents eg., carbonic anhydrase inhibitors, osmotic diuretics, high ceiling diuretics, loop diuretics, thiazide and thiazide-like diuretics, potassium-sparing diuretics, aldosterone antagonists, vasopressin and other agents as disclosed by E. K. Jackson (1996), supra. The order of administration is typically not important for purposes of this invention ie., the invention compounds can be administered to the mammal before, during, or after co-administration with the other diuretic(s).

The dosage of an invention compound to be administered to the mammal will vary according to recognized parameters and will, of course, be fitted to the individual requirements in each particular case. In general, in the case of oral administration a daily dosage of about 10 to 1000 mg per person of a compound of general formulae I, U, and/or III should be appropriate, although the range can be changed as deemed necessary by a care giver. Such dosages are generally useful for the methods disclosed herein e.g, modulation of diuresis, particularly aquaresis; prevention or treatment of edema, modulation of arterial blood pressure, antagonizing the nociceptin (ORL1) receptor in vivo or in vitro; sedating a mammal or reducing nociception in that mammal.

By the term “mammal” is meant a warm-blooded animal such as a primate, rodent, rabbit, pig, goat, sheep, horse, or other suitable model system. Preferably, the primate is chimpanzee, monkey. Typically, the primate will be a human subject in need of treatment. A preferred rodent is a mouse, hamster, gerbil or rat.

As mentioned above, the invention also features a method for making the compounds disclosed herein. Preferred methods do not produce 8-(4-isopropyl-cyclohexyl)-1-phenyl-1,3,8-triaza-spiro[4.5]decan-4-one (Compound 40) as an intermediate or final reaction product.

In one embodiment of the foregoing synthetic method, prior to step (a) of the method, the 1-phenyl-1,3,8-triaza-spiro[4.5]decan-4-one is protected in the 8-position. Typically after that step (a), the 8-position of the product is deprotected. If desired, the method can further include the step of separating the compounds into cis and trans isomers, preferably before step (d) in the method, more preferably between steps c) and d).

A more specific method of making the invention compounds is disclosed as follows.

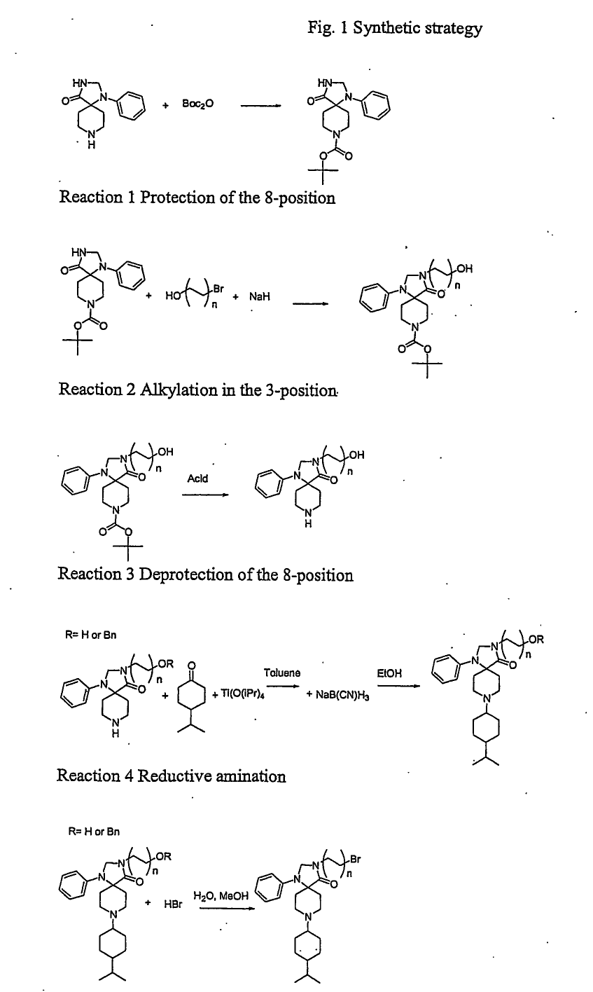

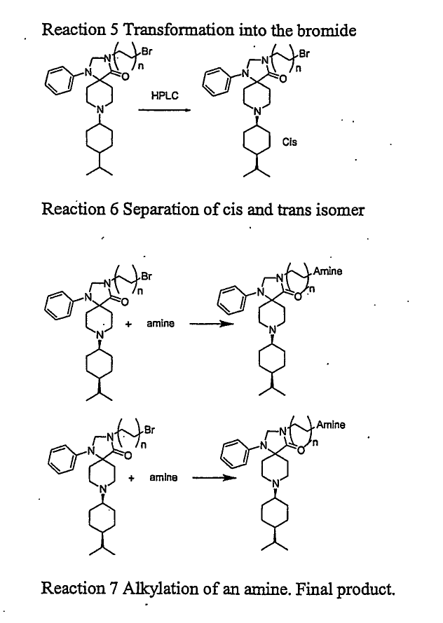

FIG. 1 provides an overview of synthetic steps used to make the compounds of Formula I and III.

As shown by FIG. 1, 1-Phenyl-1,3,8-triaza-spiro[4.5]decan-4-one (I) was Boc protected in the 8-position rendering the 3-position open for alkylation. The reaction produced the product (I) in good yield. This compound (II) was easily deprotonated with sodium hydride in DMF and alkylation with ω-bromoalkanols or ω-bromoalkyl benzyl ethers proceeded smoothly (III). Occasionally the ω-bromoalkanols gave rise to ether formation to give 8-alkyloxyalkyl-3-boc 1-phenyl-1,3,8-triaza-spiro[4.5]decan-4-one. This was avoided using the benzyl protected bromoderivative. Removal of the Boc group was performed using 50% TFA in dichloromethane and 5% EDT (IV). Omission of the EDT leads to some by-product formation. Making the 8-(4-isopropyl-cyclohexyl)-1-phenyl-1,3,8-triaza-spiro[4.5]decan-4-one derivatives (V) proceeded by reductive amination. Direct alkylation with 4-isopropyl-cyclohexyl tosylate of either the free or deprotonated amine was not always successful.

As further shown by FIG. 1, reductive amination product (V) gave rise to equal amounts of cis and trans derivatives in excellent yields. The free alcohols underwent reductive amination equally well as the benzyl ethers. It was possible to separate both the benzyl ethers and the free alcohols in a pure cis fraction and a mixture of cis and trans by prep. HPLC. The benzyl ethers were easily debenzylated using 62% hydrobromic acid. At room temperature the reaction only proceeded to the alcohol, which easily could be isolated. At 60° C. in a closed vessel the 3-bromoalkyl-8-(4-isopropyl-cyclohexyl)-1-phenyl-1,3,8-triaza-spiro[4.5]decan-4-one derivatives were formed (VI). If pure cis alcohol or cis benzyl ether was used only the cis bromide (VII) was formed. If a mixture of cis and trans was used a mixture of bromides were obtained. Also at this stage it was possible by HPLC to separate a fraction of the cis-bromide from a mixture of cis and trans bromides. The bromides were then reacted with different amine to yield the final product: a 3-substituted 8-(4-isopropyl-cyclohexyl)-1-phenyl-1,3,8-triaza-spiro[4.5]decan-4-one (VII). The reaction was sometimes slow and one to two days at room temperature or moderate heating was needed to complete it. The reaction mixture was purified on HPLC under conditions specified below. At this stage it was often possible to separate cis from trans isomers. If not, a pure cis bromide was used. The products were most often oils as TFA salts the hydrochlorides were more crystalline.

Unless otherwise specified, the following materials and methods were used to make the 3-substituted 8-(4-isopropyl-cyclohexyl)-1-phenyl-1,3,8-triaza-spiro[4.5]decan-4-one (VIII) shown in FIG. 1.

The present invention is further illustrated by the following examples. These examples are provided to aid in the understanding of the invention and are not constructed as a limitation thereof.

General Considerations: The cis and trans configurations are unambiguous assigned on the basis of the biological data: The cis being the more active. Further in RP-HPLC cis comes before trans. Unless otherwise stated the compounds were as TFA salt (see table for presumed number of TFA molecules). For purity, yield and retention times see table. Purity was based on HPLC at 254 nm. Identity was based on MS and elementary analyses. Elementary analyses were made at DB-lab in Odense Denmark

The following abbreviations were used as needed throughout the following examples: EDT: Ethanedithiol; eq. Equivalent; TFA: Trifluoroacetic acid; THF Tetrahydrofurane; Boc: tertButyloxycarbonyl; TRIS tris(2-aminoethyl)amin; ORL1-A-spiro-2-cis 8-(4-isopropyl-cyclohexyl)-1-phenyl-1,3,8-triaza-spiro[4.5]decan-4-one

Chemicals: TRIS tris(2-aminoethyl)amine; Tetraethylenepentamine; 1,4,8,11-Tetraazacyclotetradecane; Dimethylamine in ethanol 33%; Methylamine in methanol 33%, 1,4,8,11-tetraazacyclotetradecane, tetraethylenepentamine, bromoacetic acid methyl ester, 3-bromopropanol, tetraisopropyl orthotitanate, sodium cyanoborohydride, were all purchased from Fluka; 4-isopropylcyclohexanone was purchased from Lancaster. Benzyl-3-bromopropyl ether was from Aldrich. Acetonitril from SDS Tolouse France. DMF from Dupont. TFA from Halocarbon.

HPLC

Analytical:

Column Kromasil RP C8; K 100-10-C8 250×4.6 mm.

Detection 215 and 254 nm. Integration at 254 nm

Temperature: 40° C.

Flow: 1.0 ml/min

Buffers: A: 0.10% TFA in water; B: 9.90% water, 0.10% TFA 90.0% acetonitrile

Gradients
Anal1=Start 100% A 0-1.5 min 100% A 1, 5-25 min 0-50% B
Anal2=Start 40% B 0-1.5 min 40% B 1.5-15 min 40-70% B 15-20 min. 70-100% B
Anal3=Start 100% A 0-1.5 min 100% A 1, 5-25 min 0-70% B
Anal14=Start 70% B 0-1.5 min 70% B 1.5-15 min 70-100% B

Preparative:

Column Kromasil RP C8; K, 100-10-C8 250×50.8 mm.

Detection 215 and 280 nm.

Temperature ambient aprox. 20° C.

Flow: 35 ml/min

Buffers: A: 0.10% TFA in water; B: 9.90% water, 0.10% TFA 90.0% acetonitrile

Fraction size: 9 ml

Gradients:
Prep1: Start 100% A. 0-20% B in 5 min. 20-60% B in 50 min.
Prep4: Start 100% A. 0-10% B in 5 min. 10-60% B in 50 min
Prep5: Start 100% A. 0-30% B in 5 min. 30-70% B in 50 min
Prep6: Start 100% A. 0-50% B in 50 min
Prep7: Start 40% B. 40-90% B in 50 min.
Prep8: Start 30% B. 30-70% B in 50 min.

### EXAMPLE 1

### 8-(tert.Butyloxycarbonyl)-1-phenyl-1,3,8-triaza-spiro[4.5]decan-4-one (Compound 1)

1-Phenyl-1,3,8-triaza-spiro[4.5]decan-4-one (8.01 g 95%) was dissolved in dioxane (80 ml) at reflux. The flask was placed in an ice/water bath and di-tertbutylpyrocarbonate (8.442 g 1.1 eq) was added immediately and the magnetic stirring was started. The mixture was stirred for 15 min. in the bath and overnight at room temperature. The mixture was evaporated in vacuo and triturated with pentanes (100 ml) to remove excess Boc₂O. The white crystalline product was collected on a glass filter and washed with more pentanes (2×50 ml). The product was dried in vacuo to constant weight. Yield 10.62 g (92%). A sample was recrystallised from dioxane to yield a pure product mp 213.4-213.6° C. CHN: Calc. C₁₈H₂₅N₃O₃: C, 65.23; H, 7.60; N, 12.68. Found: C, 64.58; H, 7.73; N, 12.28.

### EXAMPLE 2

### 3-(3-Hydroxypropyl)-8-(tert.butyloxycarbonyl)-1-phenyl-1,3,8-triaza-spiro[4.5]decan-4-one hemihydrate (Compound 2)

Sodium hydride (60% in oil 174 mg (1.44 eq.) was washed twice with pentanes in a 50 ml centrifuge tube. 8-(tert.Butyloxycarbonyl)-1-phenyl-1,3,8-triaza-spiro[4.5]decan-4-one (1.00 g) was dissolved in DMF (10 ml) and added to the hydride. Hydrogen was evolved and the solution was allowed to react for 30 min at 22° C. It was centrifuged to precipitate unreacted NaH. 3-bromopropanol (0.340 ml 1.3 eq.) was dissolved in DMF (2 ml) and the clear solution of deprotonated spiro[4.5]decan-4-one was added. The clear reaction mixture was allowed to react overnight. After 15 min HPLC showed 60% conversion. The next day complete conversion was observed. MS confirmed identity. The mixture was evaporated to dryness. Buffer A and B (1:2 40 ml) was added. Acetonitrile was added until complete solution. The compound was purified by prep. HPLC using gradient Prep.5 to yield A 500 mg 99% pure and B 280 mg 70% pure a total of 60%. The A-fraction was crystallised from ether-pentanes. White crystals mp. 117.0-117.7° C. CHN: Calc. C₂₁H₃₁N₃O₄×½H₂O C: 63.29; H, 8.09; N, 10.54. Found: C, 63.49; H, 8.22; N, 10.19.

### EXAMPLE 3

### 3-(3-Hydroxypropyl)-1-phenyl-1,3,8-triaza-spiro[4.5]decan-4-one TFA salt. (Compound 3)

3-Hydroxypropyl-8-(tert.butyloxycarbonyl)-1-phenyl-1,3,8-triaza-spiro[4.5]decan-4-one (7 g mother liquor 60-70%) was dissolved in TFA:EDT 95:5 (50 ml) with some stirring. It was allowed to react a total of 50 min (HPLC showed complete conversion) and evaporated to dryness leaving 12 g. Water (400 ml) and ether (200 ml) and TFA (1 ml) were added to the remanens. All dissolved in either one of the phases. The aqueous phase was washed with ether (3×100 ml) and evaporated to dryness leaving 5 g. HPLC showed 85% purity. A sample of 3 g was purified on prep. HPLC using Prep6 yielding A 1106 mg 99% pure and B 300 mg 65% pure. After lyophilization A was crystalline. A sample was triturated with ethanol to yield an analytically pure sample mp 148-51° C. CHN: Calc. C₁₆H₂₃N₃O₂×C₂HF₃O₂ C, 63.29; H, 8.09; N, 10.54. Found: C, 63.49; H, 8.22; N, 10.07.

### EXAMPLE 4

### cis and cis/trans-3-(3-Hydroxypropyl)-8-(4-isopropyl-cyclohexyl)-1-phenyl-1,3,8-triaza-spiro[4.5]decan-4-one TFA salt by reductive amination (Compounds 4 and 5, respectively)

3-(3-Hydroxypropyl)-1-phenyl-1,3,8-triaza-spiro[4.5]decan-4-one TFA salt (234 mg) was dissolved in water (4 ml) and sodium hydroxide (2M 4 ml) was added and the solution was extracted with ethyl acetate (3×6 ml). The combined organic phases were dried over magnesium sulphate and the solvent removed in vacuo to yield 150 mg free base (90%). It was dissolved in refluxing toluene (10 ml) and 4-isopropylcyclohexanone (0.100 ml 1.2 eq.) was added together with tetraisopropyl orthotitanate (0.200 ml 1.17 eq.) and refluxed for 2 h preventing access of moisture. The orange solution was evaporated in vacuo and the remanens dissolved in abs.ethanol/THF (1:1 10 ml). Sodium cyanoborohydride (45 mg 1.3 eq.) was added and the solution stirred for 20 min. Hydrochloric acid (dry in dioxane 3.2 M 0.40 ml) was added to adjust pH to 5 when a drop of reaction mixture was added to wet pH-paper. Precipitation took place. The mixture was stirred for 2 h. and evaporated in vacuo. Sodium hydroxide (1M 6 ml) and ethyl acetate (8 ml) were added. Titanium salts precipitated. The biphasic mixture was centrifuged and the phases separated. The water phase was extracted with more ethyl acetate (3×8 ml). The combined organic phases were dried over MgSO₄, filtered though a little HYFLO (to remove traces of titanium salts) and evaporated to dryness yielding 213 mg (90%) of raw product 75% pure by HPLC (two peaks cis and trans) no unreacted starting material but the excess of characteristically smelling 4-isopropylcyclohexanone or 4-isopropylcyclohexanol could easily be detected. It was purified using Prep5 yielding 76 mg (25%) of pure product cis and trans. Among these it was possible to obtain 18.3 mg pure cis (15%).

### EXAMPLE 5

### 8-Boc-3-(3-benzyloxypropyl)-1-phenyl-1,3,8-triaza-spiro[4.5]decan-4-one (Compound 6)

Sodium hydride (60% in oil 1.618 g (1.35 eq.) was washed twice with pentanes. 8-(tert.Butyloxycarbonyl)-1-phenyl-1,3,8-triaza-spiro[4.5]decan-4-one (10.00 g) was dissolved in DMF (100 ml) and added to the hydride. Hydrogen was evolved and the solution was allowed to react for 30 min at 22° C. Benzyl-3-bromopropyl ether (7.39 g 1.08 eq.) was added to the solution with stirring and left overnight. HPLC showed complete conversion. The DMF is removed at 45° C. in vacuo. Ethyl acetate (200 ml) and water (100 ml) is added and the phases separated. The aqueous phase is extracted with more ethyl acetate (3×50 ml). The combined organic phases were washed with brine (2×50 ml) and dried over MgSO₄ and evaporated to constant weight. Yielding 12.84 g (89%) of a yellow glass. It was covered with pentanes (100 ml), which slowly made the glass crystallise. White waxy crystals mp 107-9° C. CHN: Calc. C₂₈H₃₇N₃O C: 70.12; H, 7.78; N, 8.76. Found: C, 69.98; H, 7.94; N, 8.73.

### EXAMPLE 6

### 3-(3-Benzyloxypropyl)-1-phenyl-1,3,8-triaza-spiro[4.5]decan-4-one Hydro chloride (Compound 7)

8-Boc-3-(3-benzyloxypropyl)-1-phenyl-1,3,8-triaza-spiro[4.5]decan-4-one (12.64 g raw) was dissolved in dichloromethane (50 ml) EDT (5 ml) was added and TFA (50 ml). The solution was allowed to react for 60 min. The solvents were removed in vacuo leaving 19 g of yellow oil. It was washed with pentanes. The TFA salt seemed to be soluble in ether hence the remaining oil was dissolved in dioxane (60 ml) and hydrochloric acid in dioxane (3.2 M 20 ml) was added. The solution was evaporated to dryness and triturated with ether (200 ml). A beige precipitate formed. The ether was decanted off and the precipitate washed with more ether. The precipitate was dried in vacuo 11.4 g (100%) HPLC showed 85% pure. MS OK. The apparently crystalline precipitate turned into a glass after some days. In the combined etheral washings a small crystalline beige precipitate formed overnight. The ether was decanted from the precipitate and it was washed with a more ether and dried. HPLC showed 98% pure. Beige crystals stable mp 174-6° C. CHN: Calc. C₂₃H₂₉N₃O₂×HCl: C, 66.41; H, 7.27; N, 10.10. Found: C, 66.14; H, 7.25; N, 9.98.

### EXAMPLE 7

### 3-(3-Benzyloxypropyl)-1-phenyl-1,3,8-triaza-spiro[4.5]decan-4-one (Compound 8)

3-(3-benzyloxypropyl)-1-phenyl-1,3,8-triaza-spiro[4.5]decan-4-one hydro chloride (5.03 g raw 80%) was dissolved in sodium hydroxide (1 M 100 ml) and ethyl acetate (200 ml) was added and the aqueous phase was extracted with ethyl acetate (3×100 ml). The combined organic phases were dried with brine (100 ml) and then over magnesium sulphate. The solvent was removed in vacuo to yield 4.27 g of free base (93%). HPLC showed identical purity when compared to the salt.

### EXAMPLE 8

### cis/trans-3-(3-Benzyloxypropyl)-8-(4-isopropyl-cyclohexyl)-1-phenyl-1,3,8-triaza-spiro[4.5]decan-4-one TFA salt (Compound 9)

3-(3-benzyloxypropyl)-1-phenyl-1,3,8-triaza-spiro[4.5]decan-4-one (3.10 g) was dissolved in toluene (30 ml) and 4-isopropylcyclohexanone (1.375 g 1.2 eq.) was added together with tetraisopropyl orthotitanate (3.00 ml 1.2 eq.) and refluxed for 2 h preventing access of moisture. The orange solution was evaporated in vacuo and the remanens dissolved in abs.ethanol (25 ml). Sodium cyanoborohydride (660 mg 1.28 eq.) was added and the solution stirred for 30 min. Hydrochloric acid (dry in dioxane 3.2 M 2.5 ml) was added to adjust pH to 5 when a drop of reaction mixture was added to wet pH-paper. Precipitation took place. The mixture was stirred for 2 h. and evaporated in vacuo. Sodium hydroxide (1M 60 ml) and ethyl acetate (40 ml) were added. Titanium salts precipitated. The biphasic mixture was filtered through HYFLO. The filter cake was washed thoroughly with ethyl acetate and sodium hydroxide and the phases separated. The water phase was extracted with more ethyl acetate (3×8 ml). The combined organic phases were dried over MgSO₄, filtered though a little HYFLO (to remove traces of titanium salts) and evaporated to dryness yielding 4.46 g free base (100%) a dark yellow oil 81% pure by HPLC (two peaks cis and trans) no unreacted starting material but the excess of characteristically smelling 4-isopropylcyclohexanone or 4-isopropylcyclohexanol can easily be detected. A sample of 1.01 g equivalent to 1.23 g of TFA salt was purified by prep. HPLC using prep7 yielded 787 mg (64%) pure material in two fractions A almost pure cis and B a mixture of cis and trans. Both were triturated with ether (1 and 3 ml respectively) to crystallise. A yielded 46.2 mg pure cis, white crystals mp 141-6° C. HPLC showed 91% purity. CHN: Calc. C₃₂H₄₅N₃O₂×C₂HF₃O₂: C, 66.11; H, 7.51; N, 6.80. Found: C, 66.89; H, 7.65; N, 6.80.

### EXAMPLE 9

### cis and cis/trans-3-(3-Hydroxypropyl)-8-(4-isopropyl-cyclohexyl)-1-phenyl-1,3,8-triaza-spiro[4.5]decan-4-one TFA salt by debenzylation (Compounds 4 and 5, respectively)

cis/trans-3-(3-Benzyloxypropyl)-8-(4-isopropyl-cyclohexyl)-1-phenyl-1,3,8-triaza-spiro[4.5]decan-4-one (2.40 g raw) was dissolved in as little methanol as possible (6 ml) in a 50 ml round bottomed flask and Hydrobromic acid (20 ml 48% in water) was added. The mixture was refluxed 5 min. HPLC showed complete debenzylation and 7% of bromide. The orange/red mixture was cooled in ice/water and neutralised with sodium hydroxide (25 ml 27%). The cold mixture was extracted with ethyl acetate (3×50 ml). The combined organic phases were dried first with brine then over MgSO₄ and the solvent removed in vacuo. Ether (40 ml) was added to the raw product and a small precipitate containing very little product was filtered off TFA (0.40 ml 1.1 eq.) was added and an oil precipitated. The ether was removed in vacuo and the raw salt was triturated with pentanes to remove traces of benzyl bromide and dried at high vacuum (0.1 mBar). Yield 1.91 mg (76%) red oil. It was dissolved in 40% acetonitrile and purified by prep. HPLC using prep8 to yield two fractions which were lyophilised: A containing exclusively cis 236 mg (19%) and B 60% trans 741 mg. Retention times were identical to the preparation by reductive amination. A was triturated with ether to give white crystals mp. 161-63°. CHN: Calc. C₂₅H₃₉N₃O₂×C₂HF₃O₂: C, 61.46; H, 7.64; N, 7.96. Found: C, 62.07; H, 7.63; N, 7.73.

### EXAMPLE 10

### cis and cis/trans-3-(3-Bromopropyl)-8-(4-isopropyl-cyclohexyl)-1-phenyl-1,3,8-triaza-spiro[4.5]decan-4-one TFA salt (Compound 9)

cis/trans-3-(3-Benzyloxypropyl)-8-(4-isopropyl-cyclohexyl)-1-phenyl-1,3,8-triaza-spiro[4.5]decan-4-one (93.5 mg raw) was dissolved in methanol (1 ml) in a 50 ml round bottomed flask and hydrobromic acid (5 ml 48% in water) was added. The mixture was refluxed 5 min. to effect debenzylation and the solvents removed in vacuo at 30° C. over 2 days, which facilitated the partial transformation into the bromide. The mixture was evaporated to dryness in vacuo. The remanens was dissolved in 40% acetonitrile in water and purified by prep. HPLC using prep8 yielding 50 mg cis/trans of these 10 mg pure cis was isolated. The fraction crystallised upon trituration with ether (0.250 ml) mp 115-122° C. CHN: Calc. C₂₅H₃₈BrN₃O×C₂BF₃O₂: C, 54.92; H, 6.66; N, 7.12. Found: C, 54.36; H, 6.70; N, 6.78.

### EXAMPLE 11

### Debenzylation and Bromodehydroxylation of Selected Compounds

cis/trans-3-(3-Benzyloxypropyl)-8-(4-isopropyl-cyclohexyl)-1-phenyl-1,3,8-triaza-spiro[4.5]decan-4-one TFA salt (41.9 mg) was dissolved in methanol (0.080 ml) with warming to 40° C. in a weighing glass. 62% Hydrobromic acid p.a. (1.00 ml) was added and the glass closed tightly. Almost instantaneously the solution became unclear and an oil separated. HPLC showed complete conversion to the alcohols and benzyl bromide. The mixture was shaken overnight at 20° C. The unclear mixture was still colourless. No triaza-spiro[4.5]decan-4-one bromide had formed. No by-products were detected by HPLC. The closed vial was warmed to 60.0° C. and became clear and slightly purple. It was left at 60.0° C. overnight. It was cooled on ice and opened carefully—a slight internal pressure was detected. HPLC showed 97.6% conversion to the bromides and 2.4% alcohols remained. No by-products were formed.

cis/trans-3-(3-Benzyloxypropyl)-8-(4-isopropyl-cyclohexyl)-1-phenyl-1,3,8-triaza-spiro[4.5]decan-4-one TFA salt (197 mg) was dissolved in methanol (0.50 ml) with warming to 40° C. in a round bottomed flask. 62% hydrobromic acid p.a. (5.00 ml) was added and the mixture refluxed for 10 min. It turned unclear and purple. HPLC showed 8% remaining alcohols 45% bromides and at least 5 by-products.

### EXAMPLE 12

### cis- and cis/trans-3-(3-Bromopropyl)-8-(4-isopropyl-cyclohexyl)-1-phenyl-1,3,8-triaza-spiro[4.5]decan-4-one TFA salt (Compound 9)

cis/trans-3-(3-Benzyloxypropyl)-8-(4-isopropyl-cyclohexyl)-1-phenyl-1,3,8-triazaspiro[4.5]decan-4-one TFA salt (52.08 mg pure) was dissolved in methanol (0.10 ml) in a weighing glass and hydrobromic acid (1 ml 62% in water) was added and the vessel closed tightly. The mixture was left overnight at 60° C. HPLC showed clean conversion to the bromides. The glass was cooled in dry ice/ethanol before opening. The mixture was dissolved in 40% acetonitrile in water and purified by prep. HPLC using prep5 yielding 26.6 mg (53%) cis/trans. Of these 6.7 mg (27%) were pure cis (91% pure).

### EXAMPLE 13

### cis and cis/trans-3-(9-Bromononyl)-8-(4-isopropyl-cyclohexyl)-1-phenyl-1,3,8-triaza-spiro[4.5]decan-4-one TFA salt

Analogous to the propyl derivative 8-(tert.Butyloxycarbonyl)-1-phenyl-1,3,8-triaza-spiro[4.5]decan-one is deprotonated with sodium hydride (1.1 eq. prewashed with pentanes) in DMF (10 ml/g of spiro compound) and reacted with 9-bromononan-1-ol (1.3 eq.) overnight. TFA (1.15 eq.) is added and the mixture is evaporated to dryness in vacuo. The remanens is washed with pentanes and subjected to deprotection by dissolving in dichloromethane (5 ml/g) EDT (5%) and TFA (5 ml/g) are added and the solution is allowed to react for 1 h. It is evaporated to dryness and washed with pentanes. It is dissolved in dioxane. Hydrochloric acid (dry in dioxane 1.1 eq.) is added and the mixture evaporated in vacuo. The remanens is triturated with ether. The free base is liberated by dissolving the product in sodium hydroxide (1M) and ethyl acetate. The aqueous phase is extracted with ethyl acetate (3 times). The combined organic phases are dried first with brine then over MgSO₄ and the solvent removed in vacuo. The base is dissolved in boiling toluene (20-30 ml/g). 4-isopropyl-cyclohexanone (1.2 eq) and tetraisopropyl orthotitanate (1.2 eq.) are added and the mixture refluxed for 2 h. protected from moisture. Most of the toluene is distilled off and the rest removed in vacuo. To the remanens is added ethanol (abs. 15 ml/g) and THF until all is in solution. Sodium cyanoborohydride (1.3 eq.) is added and the solution is stirred for 20 min. Hydrochloric acid (dry in dioxane) is added to adjust pH to 5 when a drop of reaction mixture was added to wet pH-paper. The mixture is stirred for 2 h. and evaporated in vacuo. Sodium hydroxide (1M 10 ml/g) and ethyl acetate (20 ml/g) were added. The biphasic mixture is centrifuged and the phases separated. The water phase is extracted with more ethyl acetate (3 times). The combined organic phases are dried over MgSO₄, filtered though a little HYFLO (to remove traces of titanium salts) and evaporated to dryness. The alcohol is dissolved in methanol (2-3 ml/g) and 62% hydrobromic acid (10 ml/g) is added. The vessel is closed tightly and left at 60° C. overnight. The mixture is cooled and neutralised with sodium hydroxide and small excess is added to make the aqueous phase basic. It is extracted with ethyl acetate (3 times). The combined organic phases are dried with brine and over MgSO₄. The solvent is removed in vacuo. The raw product is dissolved in 40% MeCN in water and TFA (1.1 eq) is added to secure an acidic solution. It is purified by prep. HPLC to yield a fraction A pure cis bromide and B a mixture of cis and trans.

During evaporation of the A fraction the compound can be crystallised as the acetonitrile evaporated. It can be filtered and washed with water and dried in an exicator over P₂O₅.

### EXAMPLE 14

### cis and cis/trans-3-(6-Bromohexyl)-8-(4-isopropyl-cyclohexyl)-1-phenyl-1,3,8-triaza-spiro[4.5]decan-4-one TFA salt

As in the above example only using 6-bromohexan-1-ol instead of 9-bromononan-1-ol.

### EXAMPLE 15

### cis and trans-3-(Methoxycarbonylmethyl-)-8-(4-isopropyl-cyclohexyl)-1-phenyl-1,3,8-triaza-spiro[4.5]decan-4-one TFA salt (Compound 10)

Analogous to the propyl derivative 8-(tert.Butyloxycarbonyl)-1-phenyl-1,3,8-triaza-spiro[4.5]decan-4-one is deprotonated with sodium hydride (1.1 eq. prewashed with pentanes) in DMF (10 ml/g of spiro compound) and reacted with bromoacetic acid methyl ester (1.3 eq.) overnight. TFA (1.15 eq.) is added and the mixture is evaporated to dryness in vacuo. The remainder is washed with pentanes and subjected to deprotection by dissolving in dichloromethane (5 ml/g) EDT (5%) and TFA (5 ml/g) are added and the solution is allowed to react for 1 h. It evaporated to dryness and washed with pentanes. It dissolved in dioxane. The remanens is triturated with ether. The free base is liberated by dissolving the product in sodium carbonate (saturated) and ethyl acetate. The aqueous phase is extracted with ethyl acetate (3 times). The combined organic phases are dried first with brine then over MgSO₄ and the solvent removed in vacuo. The base is dissolved in boiling toluene (20-30 ml/g). 4-isopropyl-cyclohexanol (1.2 eq) and tetraisopropyl orthotitanate (1.2 eq.) are added and the mixture refluxed for 2 h. protected from moisture. Most of the toluene is distilled off and the rest removed in vacuo. To the remanens is added ethanol (abs. 15 ml/g) and THF until all is in solution. Sodium cyanoborohydride (1.3 eq.) is added and the solution is stirred for 20 min. Hydrochloric acid (dry in dioxane) is added to adjust pH to 5 when a drop of reaction mixture is added to wet pH-paper. The mixture is stirred for 2 h. and evaporated in vacuo. Sodium carbonate (5% 10 ml/g) and ethyl acetate (20 ml/g) are added. The biphasic mixture was centrifuged and the phases separated. The water phase was extracted with more ethyl acetate (3 times). The combined organic phases are dried over MgSO₄, filtered though a little HYFLO (to remove traces of titanium salts) and evaporated to dryness. The raw product is dissolved in 40% MeCN in water and TFA (1.1 eq) is added to secure an acidic solution. It is purified by prep. HPLC and separated into cis and trans isomers.

### EXAMPLE 16

### cis-(8-(4-Isopropyl-cyclohexyl)-1-phenyl-1,3,8-triaza-spiro[4.5]decan-4-one)-acetic acid TFA salt (Compound 11)

Compound 10 (43 mg) was dissolved in ethanol (0.50 ml) in an Eppendorf tube and sodium hydroxide (2M in water 0.165 ml) was added. The solution turned yellow and was allowed to react for 18 h after which HPLC showed that reaction was complete. The reaction mixture was loaded directly on the HPLC column and purified with gradient Prep4. The pure fractions were poole and lyophilised yielding 39.5 mg (78%) of 99% purity.

### EXAMPLE 17

### Amino alkyl-8-(4-isopropyl-cyclohexyl)-1-phenyl-1,3,8-triaza-spiro[4.5]decan-4-ones

They were in general made by reacting the crude haloalkyl-8-(4-isopropyl-cyclohexyl)-1-phenyl-1,3,8-triaza-spiro[4.5]decan-4-one with a large (40-50 eq.) excess of an amine.

### EXAMPLE 18

### cis-6-Methylamino-hexyl-8-(4-isopropyl-cyclohexyl)-1-phenyl-1,3,8-triaza-spiro[4.5]decan-4-one hydrochloride, (Compound 12) trans-3-(6-methylamino-hexyl)-8-(4-isopropyl-cyclohexyl)-1-phenyl-1,3,8-triaza-spiro[4.5]decan-4-one TFA (Compound 13)

cis/trans 3-(6-Bromohexyl)-8-(4-isopropyl-cyclohexyl)-1-phenyl-1,3,8-triaza-spiro[4.5]decan-4-one was dissolved in methylamine in ethanol (2 ml 8 M 57 eq.) and allowed to react 3 days at ambient temperature. HPLC (LBH02) showed complete conversion Rt 5.8 min. (Starting material 15.3 and 15.6 min). Using another gradient (Anal1) the peak splited in two corresponding to cis and trans (24.11 and 24.38 min). The reaction mixture was purified on prep. HPLC. Gradient: Prep1. A total of 112 mg (57%) of 6-methylamino-hexyl-8-(4-isopropyl-cyclohexyl)-1-phenyl-1,3,8-triaza-spiro[4.5]decan-4-one mixture of cis and trans was obtained in three fractions a pure cis Compound 12 of 44 mg; a pure trans Compound 1312 mg and a mixture 35% cis/65% trans of 55 mg. Compound 12 was very hygroscopic and it was lyophilised from diluted hydrochloric acid to obtain the chloride salt. This was taken up in 0.50 ml methanol and precipitated with ether. Filtered on a glass filter and washed with ether an dried in vacuo to obtain 35 mg of Compound 12 which was much less hygroscopic than the trifluoroacetate. From the preparative HPLC was further isolated compounds containing an additional methylaminohexyl group Compound 14 and Compound 15. As they eluted closed to Compound 12 their purity was only 60-70%.

### EXAMPLE 19

### cis-3-Amino-propyl-8-(4-isopropyl-cyclohexyl)-1-phenyl-1,3,8-triaza-spiro[4.5]decan-4-one TFA salt (Compound 16); (Compounds 17, 18, 19 and 20)

The crude mixture of cis/trans 3-(3-bromopropyl)-8-(4-isopropyl-cyclohexyl)-1-phenyl-1,3,8-triaza-spiro[4.5]decan-4-one was dissolved in ammonia in ethanol (4.8M 3 ml) and potassium iodide (54 mg 1.2 eq.) added. It was allowed to react for five days at room temperature. The complex reaction mixture was purified by prep. HPLC gradient Prep. 1. Obtained was the desired product (Compound 16) 21.7 mg (24%). Further the trans compound (Compound 21) (21.8 mg 24%) the corresponding alcohols (Compounds 17 and 18) and some dimers (Compounds 19 and 20) in good purity.

### EXAMPLE 20

### cis-3-(6-Amino-hexyl)-8-(4-isopropyl-cyclohexyl)-1-phenyl-1,3,8-triaza-spiro[4.5]decan-4-one TFA salt (Compound 22)

cis-3-(6-Bromohexyl)-8-(4-isopropyl-cyclohexyl)-1-phenyl-1,3,8-triaza-spiro[4.5]decan-4-one TFA salt (19 mg) was dissolved in ammonia in ethanol (0.60 ml 4.9 M) and allowed to stand for three weeks. It was purified on prep. HPLC, gradient Prep4 to yield 1.2 mg (9%) 96% pure as a colourless oil.

### EXAMPLE 21

### cis-3(9-Amino-nonyl)-8-(4-isopropyl-cyclohexyl)-1-phenyl-1,3,8-triaza-spiro[4.5]decan-4-one TFA salt (Compound 23) trans-3-(9-Amino-nonyl)-8-(4-isopropyl-cyclohexyl)-1-phenyl-1,3,8-triaza-spiro[4.5]decan-4-one TFA salt (Compound 24) 1,9-bis-cis-3-nonyl-8-(4-isopropyl-cyclohexyl)-1-phenyl-1,3,8-triaza-spiro[4.5]decan-4-one TFA salt (Compound 25)

9-Bromononyl-8-(4-isopropyl-cyclohexyl)-1-phenyl-1,3,8-triaza-spiro[4.5]decan-4-one (cis/trans) 281 μmol) was dissolved in ammonia in ethanol (4.9 M 3 ml 50 eq.). The reaction was followed by HPLC. After 3 h 4% conversion was seen. After 3 days 30-40% and beginning of by-products. After 4 days the reaction mixture was purified on HPLC Prep1 yielding 51.5 mg (25%) of 3-(9-Amino-nonyl)-8-(4-isopropyl-cyclohexyl)-1-phenyl-1,3,8-triaza-spiro[4.5]decan-4-one TFA salt in three fractions 20.9 mg (20%) cis-3-(9-Amino-nonyl)-8-(4-isopropyl-cyclohexyl)-1-phenyl-1,3,8-triaza-spiro[4.5]decan-4-one TFA salt, 14.9 mg trans-9-Amino-nonyl-8-(4-isopropyl-cyclohexyl)-1-phenyl-1,3,8-triaza-spiro[4.5]decan-4-one TFA salt and a mixed fraction of 15.4 mg. In addition 36.8 mg (20%) unconverted starting material and a dimer: 1,9-bis-cis-3-nonyl-8-(4-isopropyl-cyclohexyl)-1-phenyl-1,3,8-triaza-spiro[4.5]decan-4-one TFA salt.

### EXAMPLE 22

### cis-3-(3-Dimethylamino-propyl)-8-(4-isopropyl-cyclohexyl)-1-phenyl-1,3,8-triaza-spiro[4.5]decan-4-one TFA salt (Compound 26)

cis-3-(3-Bromopropyl)-8-(4-isopropyl-cyclohexyl)-1-phenyl-1,3,8-triaza-spiro[4.5]decan-4-one TFA salt 24 mg was dissolved in ethanol (0.5 ml) in an Eppendorff tube and dimethylamine in ethanol (1.00 ml 5.6 M) was added and it was allowed to react for three weeks (one day is sufficient). It was purified with Prep4 yielding 23.4 mg (80%) of product as white crystals 99% pure.

### EXAMPLE 23

### cis-3(6-Dimethylamino-hexyl)-8-(4-isopropyl-cyclohexyl)-1-phenyl-1,3,8-triaza-spiro[4.5]decan-4-one TFA salt (Compound 27)

cis-3-(6-Bromohexyl) 8-(4-isopropyl-cyclohexyl)-1-phenyl-1,3,8-triaza-spiro[4.5]decan-4-one TFA salt (19 mg was dissolved in ethanol (0.5 ml) in an Eppendorff tube and dimethylamine in ethanol (1.00 ml 5.6 M) was added and it was allowed to react for one week. It was purified with Prep4 yielding 15.5 mg (73%) of product as white crystals 98% pure.

### EXAMPLE 24

### cis-3-(9-Dimethylamino-nonyl)-8-(4-isopropyl-cyclohexyl)-1-phenyl-1,3,8-triaza-spiro[4.5]decan-4-one TFA salt Bx 38.16; (Compound 28)

cis-3-(9-Bromononyl)-8-(4-isopropyl-cyclohexyl)-1-phenyl-1,3,8-triaza-spiro[4.5]decan-4-one TFA salt (150 mg purified) was dissolved in ethanol (3 ml) and dimethylamine in ethanol (5.0 ml 5.6 M 130 eqv) was added and it was allowed to react for one day. After 5 min 5% conversion was detected. It was evaporated to dryness dissolved in 40% acetonitrile and acidified with TFA (0.20 ml). It was purified with Prep4 yielding 150 mg (83%) of product as a yellow oil which slowly crystallised (90% pure). Some was triturated with dioxan which crystallised the compound and gave purity of 99% white crystalline material mp 131-3° C. CHN: Calc. C₃₃H₅₆N₄O×2C₂HF₃O₂×2H₂O: C, 55.23; H: 7.92; N, 7.10. Found: C, 56.53; H, 7.51; N, 6.82. Ether also crystallise the material but did not have a purifying effect.

### EXAMPLE 25

### cis-3-(7-Aminoethyl-4,7,10-triazadecan)-8-(4-isopropyl-cyclohexyl)-1-phenyl-1,3,8-triaza-spiro[4.5]decan-4-one TFA salt (Compound 29)

cis-3-(3-Bromopropyl)-8-(4-isopropyl-cyclohexyl)-1-phenyl-1,3,8-triaza-spiro[4.5]decan-4-one TFA salt (125 mg) was dissolved in ethanol (2 ml) in and TRIS (2.00 ml 64 eqv.) was added and it was allowed to react for one day. It was purified with Prep1 yielding 139 mg (65%) of product as a white powder 93% pure.

### EXAMPLE 26

### cis-3-(10-Aminoethyl-7,10,13-triazamidecan)-8-(4-isopropyl-cyclohexyl) 1-phenyl-1,3,8-triaza-spiro[4.5]decan-4-one TFA salt (Compound 30)

cis-3-(6-Bromohexyl)-8-(4-isopropyl-cyclohexyl)-1-phenyl-1,3,8-triaza-spiro[4.5]decan-4-one TFA salt (19 mg) was dissolved in ethanol (0.5 ml) in an Eppendorff tube and TRIS (1.00 ml 220 eq.) was added and it was allowed to react for one week. It was purified with Prep4 yielding 14 mg (45%) of product as colourless oil 99% pure.

### EXAMPLE 27

### cis-3-(13-Aminoethyl-10,13,16-triazahexadecan)-8-(4-isopropyl-cyclohexyl)-1-phenyl-1,3,8-triaza-spiro[4.5]decan-4-one TFA salt (Compound 31) trans-(13-Aminoethyl-10,13,16-triazahexadecan)-8-(4-isopropyl-cyclohexyl)-1-phenyl-1,3,8-triaza-spiro[4.5]decan-4-one TFA salt (Compound 32)

cis/trans-3-(9-Bromononyl)-8-(4-isopropyl-cyclohexyl)-1-phenyl-1,3,8-triaza-spiro[4.5]decan-4-one TFA salt (36.8 mg) was dissolved in ethanol (0.5 ml) in an Eppendorff tube and TRIS (0.50 ml 56 eq.) was added and it was allowed to react for three days and no more starting material was present. It was purified with Prep4 yielding 18.4 mg (31%) of product as oils. It was in three fractions 9.0 mg Compound 31 (93% pure), 3.3 mg Compound 32 (99% pure) and 6.1 mg as a mixture.

### EXAMPLE 28

### cis-8-(4-Isopropyl-cyclohexyl)-3-(4,7,10,14,17-pentaazaheptadecyl)-1-phenyl-1,3,8-triaza-spiro[4.5]decan-4-one TFA salt (Compound 33)

cis-3-(3-Bromopropyl)-8-(4-isopropyl-cyclohexyl)-1-phenyl-1,3,8-triazaspiro[4.5]decan-4-one TFA salt (48 mg 85% pure) was dissolved in DMF (1.0 ml) in an Eppendorff tube. Tetraethylenpentamine (1.0 ml) were added and the solution left one week at room temp. HPLC showed complete conversion. The solution was diluted with water (10 ml) and acidified with TFA (3 ml) and purified with Prep4 yielding 36.6 mg (36%) Compound 33 (91% pure)

### EXAMPLE 29

### cis-8-(4-Isopropyl-cyclohexyl)-3-(7,10,14,17,20-pentaazaeicosanyl)-1-phenyl-1,3,8-triaza-spiro[4.5]decan-4-one TFA salt (Compound 34)

cis-3-(6-Bromohexyl) 8-(4-isopropyl-cyclohexyl)-1-phenyl-1,3,8-triaza-spiro[4.5]decan-4-one TEA salt (135 mg) was dissolved in ethanol (4 ml). Tetraethylenpentamine (2.0 ml) was added and the solution left one day at room temp. HPLC showed complete conversion. The solution was diluted with water (10 ml) and acidified with TFA (3 ml) and purified with Prep4 yielding 198.8 mg (78%) Compound 34 (95% pure).

### EXAMPLE 30

### cis-3-(7-Aminoethyl-4,7,10-triazadecan)-8-(4-isopropyl-cyclohexyl)-1-phenyl-1,3,8-triaza-spiro[4.5]decan-4-one TFA salt (Compound 35)

cis-3-(9-Bromononyl)-8-(4-isopropyl-cyclohexyl)-1-phenyl-1,3,8-triaza-spiro[4.5]decan-4-one TFA salt (188 mg) was dissolved in ethanol (4 ml) and tetraethylenepentamine (2.00 ml 38 eqv.) was added and it was allowed to react for one day and no more starting material was present. Water and TFA (until acidic pH) was added. It was purified with Prep4 yielding 228 mg (66%) of product as yellow deliquescent powder 75% pure.

### EXAMPLE 31

### cis-8-(4-Isopropyl-cyclohexyl)-1-phenyl-3-[3-(1,4,8,11-tetraaza-cyclotetradec-1-yl)-propyl]-1,3,8-triaza-spiro[4.5]decan-4-one TFA salt Bx 05.94 and (Compound 36) cis-3-Allyl-8-(4-isopropyl-cyclohexyl)-1-phenyl-1,3,8-triaza-spiro[4.5]decan-4-one TFA salt (Compound 37)

cis-3-(3-Bromopropyl)-8-(4-isopropyl-cyclohexyl)-1-phenyl-1,3,8-triaza-spiro[4.5]decan-4-one TFA salt (125 mg) and 1,4,8,11-tetraazacyclotetradecane (500 mg 12 eq.) were dissolved in isopropanol:ethanol (1:1 8 ml) with warming. This amine is more soluble in isopropanol than in ethanol. It was heated to 70° C. for 1 day in a closed vessel giving a clear solution. Upon cooling unreacted amine crystallised. HPLC showed complete conversion and approx. 63% product. Water (20 ml), TFA (1 ml) for acidification and acetonitril (ca 20 ml) until complete dissolution were added and the solution purified by Prep4 yielding 139.9 mg (56%) of Compound 36 (90% pure) as a white powder. A more apolar fraction contained the cis 3-allyl-8-(4-isopropyl-cyclohexyl)-1-phenyl-1,3,8-triaza-spiro[4.5]decan-4-one TFA salt Compound 371.5 mg (83% pure).

### EXAMPLE 32

### cis-8-(4-Isopropyl-cyclohexyl)-1-phenyl-3-[6-(1,4,8,11-tetraaza-cyclotetradec-1-yl)-hexyl]-1,3,8-triaza-spiro[4.5]decan-4-one TFA salt (Compound 38)

cis-3-(6-Bromohexyl)-8-(4-isopropyl-cyclohexyl)-1-phenyl-1,3,8-triaza-spiro[4.5]decan-4-one TFA salt (135 mg) was dissolved in isopropanol (10 ml) and 1,4,8,11-tetraazacyclotetradecane (700 mg 16 eq.) was added. This amine is more soluble in isopropanol than in ethanol. It was heated to 70° C. for 1 day in a closed vessel. Upon cooling unreacted amine crystallised. Water (20 ml), TFA (1 ml) for acidification and acetonitril (ca 20 ml) until complete dissolution were added and the solution purified by Prep1 yielding 104.8 mg (41%) of Compound 38 (85% pure) as a reddish powder

### EXAMPLE 33

### cis-8-(4-Isopropyl-cyclohexyl)-1-phenyl-3-[9-(1,4,8,11-tetraaza-cyclotetradec-1-yl)-nonyl]-1,3,8-triaza-spiro[4.5]decan-4-one TFA salt (Compound 39)

cis-3-(9-Bromononyl)-8-(4-isopropyl-cyclohexyl)-1-phenyl-1,3,8-triaza-spiro[4.5]decan-4-one TFA salt (188 mg) was dissolved in ethanol (4 ml) and 1,4,8,11-tetraazacyclotetradecane (404 mg 7 eq.) dissolved in isopropanol (5 ml) was added and it was allowed to react for overnight at 70° C. HPLC showed complete conversion. Water (10 ml) was added and TFA to acidification and acetonitrile to solution (ca 10 ml) It was purified with Prep4 yielding 223.6 mg (64%) of product as yellow oil 79% pure.

### EXAMPLE 34

### Properties of polar tailed 1,3,8-triaza-spiro[4.5]decan-4-ones

The oral bioavailability of several compounds was investigated in Sprague-Dawley rats. The compounds were administered i.v. and p.o. bolus to two rats in a cross-over design. Blood samples were collected in the time interval from 0 to 300 min (except Compound 280 to 1140 min). The concentrations of the compounds in the obtained plasma were quantified by LC/MS/MS using an external calibration curve. Plasma concentration vs. time data were analyzed by non compartmental modelling and the dose corrected AUC's from the i.v. and p.o. administered rats used to calculate the oral bioavailability.

The following materials and methods were used in this example.

A. Chemicals & Materials

The water used for these experiments was of highest quality obtained from a reversed osmosis primary system in combination with a Milli-Q water secondary treatment system (Millipore, Bedford, Mass., USA). Methanol was super gradient quality obtained from Labscan Ltd. (Dublin, Ireland). Formic acid p.a. (98-100%), was obtained from Merck (Darmstadt, Germany). Heptafluorobutyric acid, HPLC grade was obtained from Pierce (Rockford, Ill., USA). EDTA stabilised plasma from rat (Sprague-Dawley) was obtained from Harlan Sera Lab Ltd. (Loughborough, UK). Blood samples were collected in potassium EDTA coated microtainers from BD Vacutainer Systems (Plymouth, UK). Sample preparation by ultra filtration was performed using Microcon centrifugal filter devices with a molecular weight cut off of 3000 obtained from Millipore (Bedford, Mass., USA).

B. Instrumentation

The LC/MS/MS analysis was performed on a Waters Alliance 2790 HPLC instrument in combination with a Quattro Ultima mass spectrometer from Micromass (Manchester, UK). Both the LC and MS were controlled by MassLynx 3.5 software. The LC separations prior to MS/MS detection were performed on an XTerra MS C₁₋₈ (3.0×50 mm), 3.5 μm particles, (Waters, Milford, Mass., USA).

C. Animals

Twenty male Sprague-Dawley rats (approx 350 g) were obtained from M&B (Denmark) and catheters were inserted into the femoral vein and artery during Hypnorm®-Dormicum® anaesthesia. After surgery, the rats were allowed to reconstitute for five days before drug administration was initiated.

D. Compound and Dose Levels

All compounds (Table 1) were obtained as TFA salts, dissolved in 5% ethanol in water and administered as bolus injections in volumes of 1 mL/kg both for i.v. and p.o. dosing. The dosing solutions used for i.v. and p.o. administrations were diluted (100 and 500 times, respectively) in 0.5% formic acid in water and analysed by LC/MS/MS for their content of drug substance. The concentrations of drug substance in the dosing solutions were calculated from the responses of standards in the range from 1 to 1000 mM and the respective administered doses displayed in Table 1.

TABLE 1

Dose (nmol/kg)

| Substance | i.v. | p.o. |
|---|---|---|
| (Compound 40) | 82 | 519 |
| (Compound 11) | 33 | 400 |
| (Compound 28) | 84 | 423 |
| (Compound 29) | 181 | 1110 |
| (Compound 35) | 92 | 410 |
| (Compound 39) | 101 | 434 |
| (Compound 36) | 106 | 400 |

Each compound was administered to two animals in a cross-over study design as i.v. and p.o. bolus. The p.o. dose was administered to rats fasted for a 12 hours period prior to drug administration. The animals were allowed to rest for 48 hours in between drug administrations. After the first experiment the rats received a blood transfusion from a litter mate.

Prior to compound administration (5 min) the rats received 500 IU heparin as an i.v. bolus and a control blood sample was obtained. After drug administration blood samples of approx. 250 μL were collected at the time points listed in Table 2. The blood samples were stored on ice until centrifugation for 5 min at 10.000×g (4° C.) and the plasma (100 μL) were transferred to 1.5 mL polypropylene tubes and stored at −20° C. until sample preparation and LCMS/MS analysis.

TABLE 2

| Route | Scheduled blood sampling time points (min) |
|---|---|
| i.v. | B.D., 5, 15, 30, 60, 120, 180, 240, and 300. |
| p.o. | B.D., 10, 30, 50, 80, 120, 180, 240, and 300. |
| | ^{#}B.D., 60, 120, 180, 240, 300, 360, 480, and 1440. |

Table 2 shows a blood sampling scheme for the i.v. and p.o. administered rats. B.D.: Before dose, #: Due to slow absorption the time schedule for drug substance Compound 28 was modified.

E. Sample Preparation and LC/MS/MS Analysis

The plasma samples were thawed on ice, mixed with 100 μL 1% (v/v) formic acid and transferred to microcon YM-3 filter units. The samples were then centrifuged for 1 hr at 8.300×g at room temperature. The filtrates were collected in 250 μL autosampler vials and stored at 4° C. until injection and analysis by LC/MS/MS. The LC/MS/MS settings for the various compounds are listed in Appendix 1.

F. Data Analysis

The plasma concentration of the compounds were calculated from the area related to an external calibration curve obtained from the analysis of blank plasma spiked with the drug substance and subjected to sample preparation and LC/MS/MS analysis. The calibration curve covered the concentrations from 5 to 5000 nM.

The plasma concentrations versus time data from each compound was used for pharmacokinetic modelling in WinNonLin 3.5 (Pharsight, Mountain View, Calif., USA) using non-compartmental analysis and the value of the parameters Fₚₒ, and t_{1/2}, are reported in Table 6.

In total seven compounds were analysed. The data obtained on Compound 40 and Compound 11 were based on responses below the lowest calibration concentration (5 nM) and therefore should be used as guidance only. The low responses of the compounds were found to be caused by a suppression effect in plasma originating from the pharmacokinetic animals. The suppression was estimated to 86% for both compounds when compared to the responses from spiked blank plasma obtained from Harlan Sera Lab. The parameters affected by the plasma concentration were therefore mathematically corrected using a factor of 0.14.

### EXAMPLE 35

### Assay of 3-substituted 8-(4-isopropyl-cyclohexyl)-1-phenyl-1,3,8-triaza-spiro[4.5]decan-4-ones for diuretic activity

The following assay was used to test compounds for diuretic activity.

Male SD/TAC rats (M&B, Denmark) weighing approximately 250 g were used fore the experiment. The animals were housed individually in Macrolon type III cages under controlled conditions (20° C., 55-85% humidity) following a 12:12-h light:dark cycle with light on at 6 am. The animals were given access to food (Altromin no 1324 diet, Chr. Petersen, Ringsted, Denmark) and domestic quality tap water ad libitum. All animals were given a minimum of 4 days acclimatization period before entering the experiment. During metabolic measurements the rats were housed in metabolic cages (type Ricambi 3700M0-000, Scandidact, Denmark). Other conditions remain the same, except access to food was denied during the experiment. The animals were sacrificed at the end of the experiment.

The animals were given access to drinking water ad libitum all through the experiment but were fasted the afternoon before the experiment. On the day of experiment the animals were housed in the metabolic cages (no access to food) for one hour before the experiment was commenced. The test compounds were administered either s.c. or p.o. in a volume of 5 ml/kg or i.v. in 1 ml/kg

Before given the injection of test compound or vehicle the animals bladder were emptied by palpation. After injection the animals were put back in the metabolic cages. The cages were fitted with clean, tarred urine sample tubes. Two hours and four hours after test compound administration the animals bladder were again emptied by palpation and the amount of urine collected was measured gravimetrically.

Table 4, below, shows the diuretic effect of selected compounds in the assay.

TABLE 4

| | | SC Dose at | | PO Dose at | Lovest Dose at which | | |
|---|---|---|---|---|---|---|---|
| | | which max | | which max | increased diuresis is | | |
| | | diuresis is | | diuresis is | achieved nMol/kg. | | |
| | SC max | achieved | PO max | achieved | I.e. >1.5 | | |
| Compound | effect. | nMol/kg | effect. | nMol/kg | IV | SC | PO |
| (Compound 41) | 2.0 | 1000 | 1.6 | 30 | 10 | 1000 | 30 |
| (Compound 40) | 3.3 | 3000 | NI | | 1000 | 1000 | |
| (Compound 10) | 1.9 | 1000 | NI | | NT | 1000 | |
| (Compound 11) | 2.0 | 1000 | NI | | 10 | 1000 | |
| (Compound 12) | 2.2 | 3000 | NI | | NT | 3000 | |
| (Compound 14) | 2.5 | 100 | NT | | NT | 100 | |
| (Compound 16) | 2.5 | 3000 | 1.9 | 1000 | NT | 1000 | 1000 |
| (Compound 23) | NI | | NI | | NT | | |
| (Compound 25) | 2.5 | 1000 | 2.5 | 1000 | 10⁻⁵ | 10 | 10 |
| (Compound 31) | 4.4 | 100 | NI | | NT | 10 | |
| (Compound 32) | 2.5 | 100 | NI | | NT | 100 | |
| (Compound 26) | NI | | NI | | NT | | |
| (Compound 28) | NI | | 2.3 | 100; 1000 | 1 | | 100 |
| (Compound 29) | 4.8 | 1000 | 1.9 | 3000 | 10 | 10 | 1000 |
| (Compound 36) | 3.5 | 100 | NI | | NT | 100 | |
| (Compound 27) | NI | | NI | | NT | | |
| (Compound 35) | 3.3 | 300 | 1.8 | 300 | 100 | 30 | 300 |
| (Compound 39) | 3.3 | 1000 | NI | | 100 | 100 | |
| (Compound 34) | 4.9 | 100 | 2.0 | 0.1; 10 | 0.0001 | 10 | 0.1 |
| (Compound 33) | 3.3 | 1000 | 1.6 | 1000 | NT | 0.1 | 1000 |
| (Compound 36) | 4.9 | 1000 | 2.4 | 1000 | 10 | 0.1 | 1000 |
| (Compound 38) | NT | | NI | | NT | | |

NI No increase
NT Not Tested
The effect number is the ratio of the measured urine from rats receiving the compound compared to a control-group receiving vehicle. I.e. 1 is normal diuresis. The ratio is only considered increased if greater than 1.5. Maxium test dose is 3000 nMol/kg. Usual steps between doses is one decade i.e. 0.1, 1, 10, 100, 1000 or 3, 30, 300, 3000.

### EXAMPLE 36

### Binding of Nociceptin and 3-Substituted 8-(4-isopropyl-cyclohexyl)-1-phenyl-1,3,8-triaza-spiro[4.5]decan-4-ones to the hORL1 Receptor

Briefly, the assay was conducted by quantifying inhibition of [³H]nociceptin binding by a polar tailed 1,3,8-triaza-spiro[4.5]decan-4-one. The assay employed membranes isolated from HEK293 cells stably expressing cloned hORL1 receptors. Additional information about the assay has been disclosed in.

A. Human Orphanin Receptor (hORL1) Cell Line

hORL1 cells were grown to near confluence in MEM supplemented with 10% FCS in 175 cm² culture flasks at 37° C., 5% CO₂ and 100% humidity. Cells were harvested in ice-cold PBS and centrifuged at 1000×G for 10 min. at 4° C. The sedimented cells were lysed in a 2.5 ml dist. H₂O/culture flask at 0° C. for 30 min. and centrifuged at 50,000×G, 4° C. for 45 min. The membrane pellet was taken up in binding buffer (50 mM HEPES, 1 mM EDTA, 10 mM MgCl₂, pH 7.4) supplemented with 10% sucrose and stored at −80° C. until use.

For stimulation of cAMP formation (see Example 37, below), cells were seeded in 96-well microtiter plates (Nunc) at a density of 2,500 cells/well (˜7,580 cells/cm²) and grown for 3 days before use under the same culture conditions as mentioned above.

B. The hORL1 Membrane Receptor Binding Assay

hORL1 membrane receptors (10 μgₚᵣₒₜₑᵢₙ/assay) produced in part A (above) were incubated for 60 min. at 25° C. in a total volume of 100 μl binding buffer (50 mM HEPES+1 mM EDTA+10 mM MgCl₂, pH 7.4) supplemented with 1% BSA (to avoid ligand depletion) together with 1.2 nM [³]nociceptin either alone (Total binding), in the presence of 1 μM nociceptin (Non-specific binding) or 5 concentrations of compound.

The binding reaction was stopped by vacuum filtration on a Packard Cell Harvester onto 96-well UniFilter® GF/C™ pre-soaked at least 30 min. before use in 0.5% polyethyleneimine (PEI) followed by 3 washes with ice-cold binding buffer. Filters were dried for 90 min. at 60° C. before adding 50 μl scintillation fluid (Ultima Gold cat. No. 6013329, Packard). The filter-bound radioactivity (˜membrane-bound) was measured in a TopCount™ (Packard Life Sciences) scintillation counter.

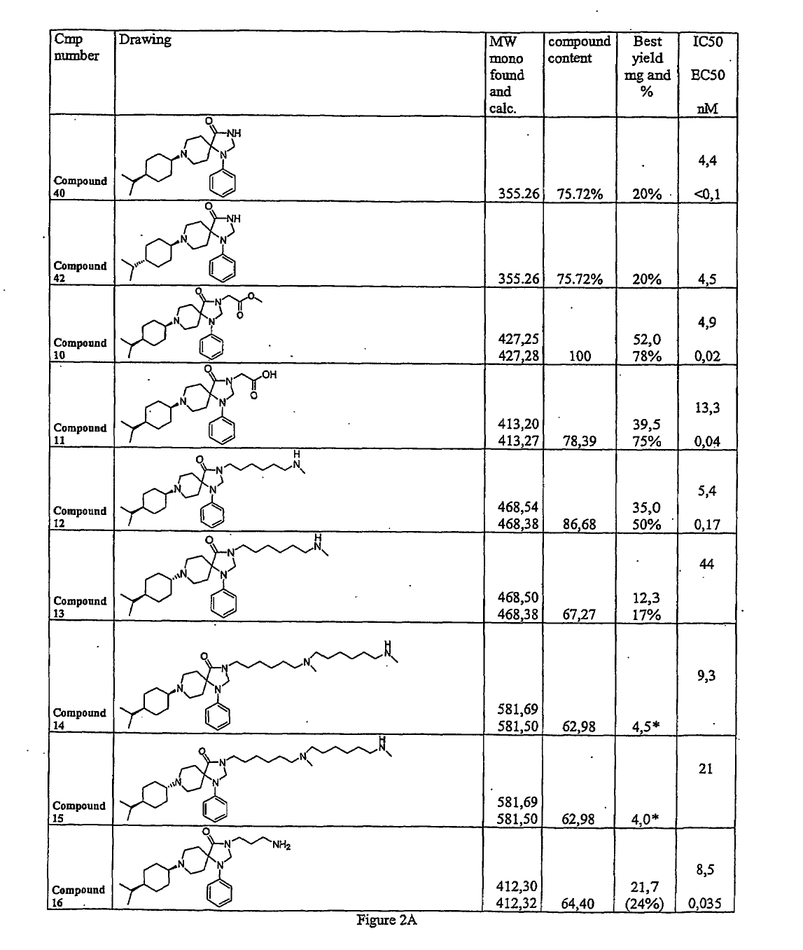

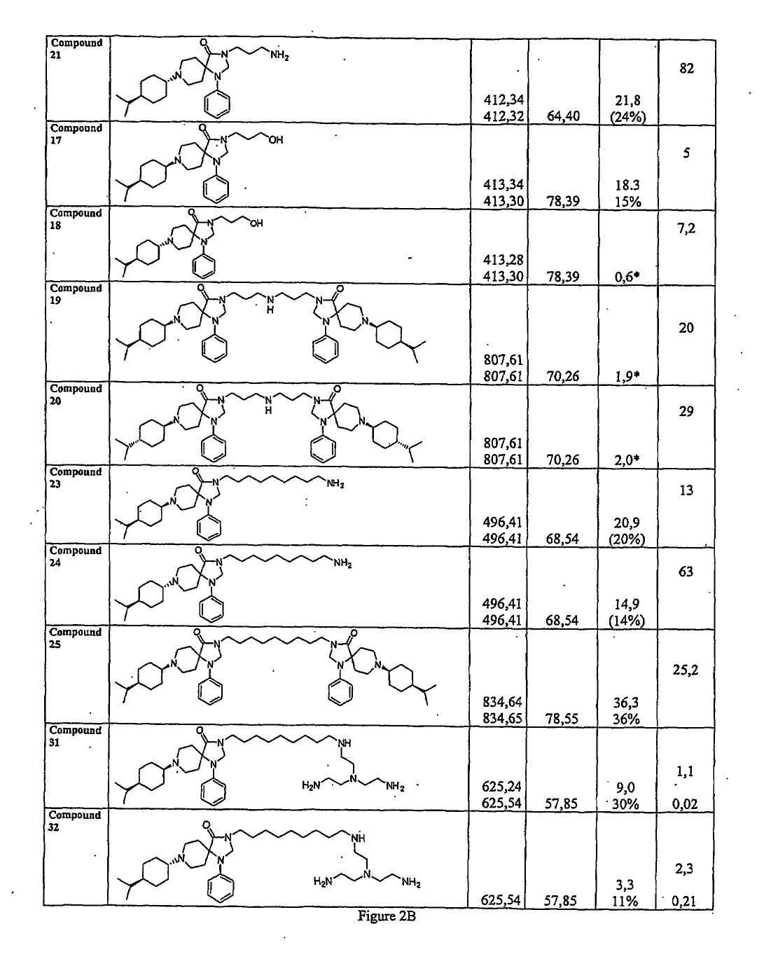

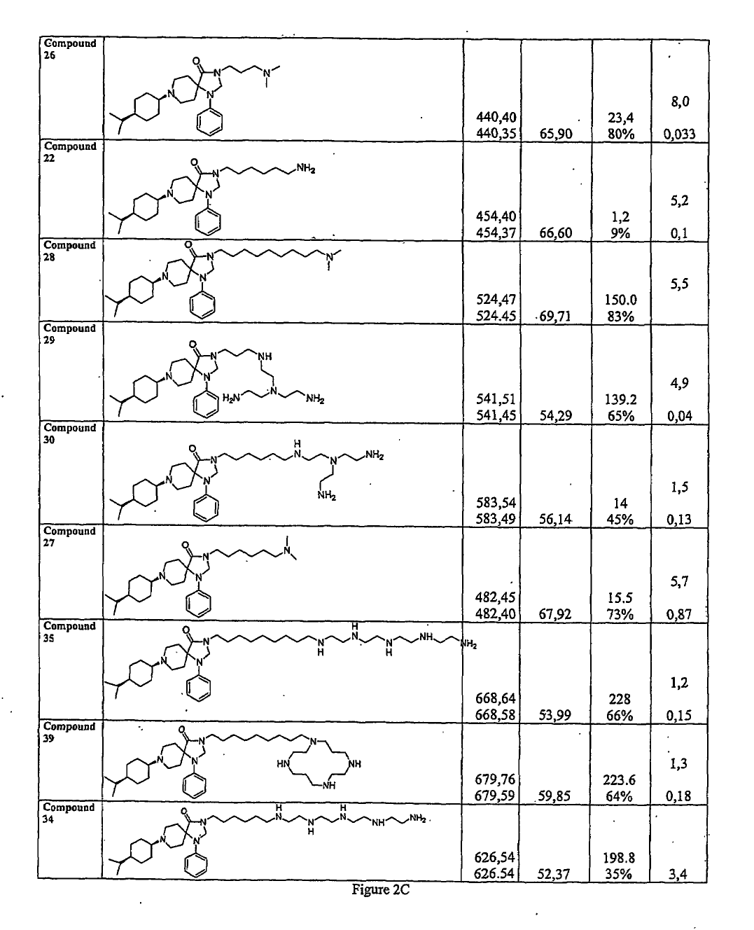

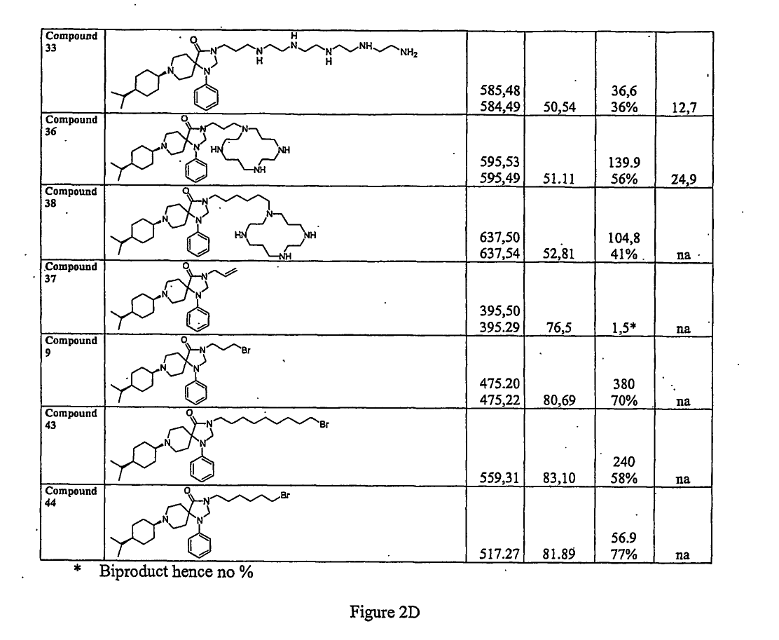

The binding data for several polar tailed 1,3,8-triaza-spiro[4.5]decan-4-ones are shown in FIGS. 2A-D.

The following materials and methods were used, as needed, in this example: hORL1 cells (an HEK293 cell line stably transfected with human ORL1 orphanin receptor, Cat #RBHORLC) are from Receptor Biology, Inc. (Canada). Minimum Essential Medium (MEM) with Earl's salts, (GibcoBRL cat. No. 21090022) and Foetal Calf Serum (cat No. 10106-163, batch No. 200249 52) are from Life Technologies. Nociceptin [Leucyl-3,4,5-³H], 87.7 Ci/mmol (Cat No. NET1130) and cAMP [¹²⁵] FlashPlate® Assay kit (Cat #SMP001A) are from NEN Life Science Products, Belgium. Culture flasks and plates are from Nunc A/S, Denmark. All other chemicals are from standard commercial sources or where made as described herein.

### EXAMPLE 37

### Effect of Polar Tailed 8-(4-isopropyl-cyclohexyl)-1-phenyl-1,3,8-triaza-spiro[4.5]decan-4-ones on Forskoline Induced cAMP Formation in HEK293 Cells

A. hORL1 Receptor Mediated Inhibition of Forskoline-Induced CAMP Formation

On the day of analysis growth medium was removed, and the hORL1-HEK293 cells were washed twice in Dulbeccos Phosphate Buffered Saline™ (D-PBS) containing 6 mM glucose at 37° C. Cells were then incubated at 37° C. for 20 min. in the same medium supplemented with 1 μM forskoline+2 mM IBMX, a phosphodiesterase inhibitor, and increasing concentrations of compound. The reaction was stopped by addition of ice-cold 20 μl 0.50 M HCl and incubation on ice for further 20 min. 20 μl of the acid extract was used for determination of cAMP by the FlashPlate™ technique, and another 20 μl were used for determination of protein content.

B. cAMP Efficacy in CHO Cells Expressing Cloned hORL1 Receptor

The effect of compound is measured as inhibition of forskoline-induced stimulation of cAMP formation in HEK293 cells stably expressing cloned hORL1 receptors. Compound 41 was found to inhibit forskoline-induced cAMP formation in HEK293 cells expressing cloned hORL1 receptors. See also for more information.

The following general methods were used as needed to perform the examples described in this application

1. Protein Determination

The protein contents of test samples were measured according to standard methods.

2. Data Analysis

Data from the displacement experiments were fitted to the equation:

f==[(Total−ns)/(1+s/IC₅₀)]+ns

where Total is the total bound radioactivity at concentration s of labelled ligand, ns is non-specific binding, and IC₅₀ is the concentration of test compound reducing specific binding (Total−ns) to 50% of maximum specific binding.

Data from the cAMP experiments are treated in an analogue way and fitted to:

f=[(cAMP_{1 μM fosk}−cAMP₀)/(1+s/EC₅₀)]+cAMP₀

### EXAMPLE 38

### Effects of Certain 3-Substituted 1,3,8-triaza-spiro[4.5]decan-4-ones

As discussed, it is an object of this invention to improve oral bioavailability of 1,3,8-triaza-spiro[4.5]decan-4-ones by adding a polar tail and at the same time reduce the CNS effects. The extend of the CNS effects were determined by the following assays.

A1. Gridshock Test

The gridshock test has been generally described. See Crawley, J. N., Brain Res., 835 (1999) 18; and Wainai, T., et al. Neuroreport, 12 (2001) 3169. It was employed in a modified version to evaluate pain threshold in mice as follows.

The gridshock apparatus consists of a plexiglas cage (L 33 cm, W 23 cm, H 15 cm) with a grid floor. Each string in the grid is 0.1 mm wide, and they are placed with a distance of 4.4 mm in between. Through the grid, electric shock is delivered to the feet of the mouse with the current increasing over time. The cage was designed with a microphone in the lid and the current is cut off as soon as the mouse makes a sound. The pain response in the gridshock test was measured as the current (mA), where the mouse starts to feel pain, i.e. the mouse makes a sound.

Before the determination of pain threshold was started, the mouse was weighted, to be able to inject the correct dose of test compound (3-substituted 1,3,8-triaza-spiro[4.5]decan-4-one). After administration of test compound or vehicle the mouse was placed in macrolon type 2 cages along with the four other mice belonging to the particular group. The test was performed by placing each mouse in the plexiglas cage of the gridshock apparatus and then the current was turned on. Each mouse was individually tested for pain threshold just before and 15, 30 and 60 minutes after injection of test compound or vehicle. The response 15, 30 or 60 minutes after injection was divided with the response before injection, to give a relative response.

The upper limit, which is the highest current applied to the mice during the test, is individual and was calculated as twice the value determined before injection. If the value before injection was lower than 0.200 mA or higher than 0.400 mA the mouse was excluded from the experiment. For every two test groups one vehicle group was tested. n=5 in both vehicle and test group. No persons, other than the investigator, were allowed in the room, and there should be quiet during the test. It should be the same investigator performing all the tests. On the day of testing the mice should not be moved from their home cage, before the test is initiated.

Before the eight test compounds were tested, the gridshock test was validated using morphine and nociceptin as controls.

The injected compounds (TFA-salts) were all pH-adjusted to be between 5-7.8, using NaOH or citric acid. The test compounds (3-substituted 1,3,8-triaza-spiro[4.5]decan-4-ones) and vehicle were administered i.v. in the tail of the mouse. The volume injected i.v. was 100 μL/

The animals used were male NMRI mice (M&B Taconic) weighing 18-25 g. The age of the mice were four weeks.

The maximal tolerated dose was defined as the highest dose administered, without statistical significant deviation from the vehicle group. The locomoter activity test is explained in more detail below.

A2. Data Analysis

The data obtained in the gridshock test was treated statistically by a two-way analysis of variance (ANOVA) and a post-hoc test. The post-hoc test used was The Fisher Least Significant difference (LSD) method. The maximal tolerated dose was identified using the statistic analysis mentioned above. The vehicle group used in the statistical analysis of each test compound was a pooled group containing all the vehicle groups related to the compound in question. The pooling implies that no statistical significant difference is present between the vehicle groups

A3. Gridshock Test Results

Nociceptin was tested i.v. in the doses 30; 3000 and 10000 mol/kg. No statistical significant difference in the gridshock response was observed.

The effect of TFA was tested in the dose 50000 nmol/kg i.v. No statistical significant difference in the gridshock response for 50000 nmol/kg TFA i.v. was observed.

Compound 40 was tested i.v. in the doses 3, 300, 1000, and 2000 nmol/kg. After injection of the dose 3000 nmol/kg, the mice were highly sedated and performing the test was worthless. Therefore it was not possible to test higher doses than 2000 nmol/kg. Statistical significant analgesia was observed at the doses 1000 and 2000 nmol/kg.

Compound 11 was tested i.v. in the doses 30, 3000 and 10000 mmol/kg. Higher doses were not tested, due to problems concerning the dissolubility of the compound. No statistical significant difference in the gridshock response was observed.

Compound 28 was tested i.v. in the doses 30, 3000 and 10000 nmol/kg. At the dose 30000 nmol/kg the mice died immediately after injection. Therefore higher doses than 1000 nmol/kg were not tested. Statistical significant analgesia was observed at the dose 10000 nmol/kg at 15 and 30 minutes.

Compound 29 was tested i.v. in the doses 1, 100, 300 and 1000 mol/kg. At the dose 2000 mmol/kg, the mice died approximately 1 minute after injection. Therefore higher doses than 1000 nmol/kg were not tested. Statistical significant analgesia was observed at the dose 1000 nmol/kg at 15 and 30 minutes.

Compound 35 was tested i.v. in the doses 1, 100, 300 and 1000 nmol/kg. After injection of the dose 2000 mmol/kg, the mice were highly sedated and had difficulties in breathing. Besides this, the mice had convulsions in the legs and died after approximately 2 minutes. Therefore higher doses than 1000 nmol/kg were not tested. Statistical significant analgesia was observed at the doses 300 and 1000 nmol/kg.

Compound 39 was tested i.v. in the doses 3, 300, 1000, 3000 and 5000 nmol/kg. After injection of the dose 10000 nmol/kg, the mice had difficulties in breathing and had convulsions in the legs. They died approximately 2 minutes after injection. Therefore higher doses than 5000 nmol/kg were not tested. Statistical significant analgesia was observed at the dose 5000 mmol/kg.

Compound 34 was tested i.v. in the doses 1, 100, 300 and 1000 mol/kg. After injection of the dose 2000 nmol/kg, the mice were sedated and had difficulties in breathing. They died approximately 2 minutes after injection. Therefore higher doses than 1000 nmol/kg were not tested. No statistical significant difference in the gridshock response was observed.

Compound 36 was tested i.v. in the doses 3, 300 and 1000 nmol/kg. After injection of the dose 2000 nmol/kg, the mice had difficulties in breathing and had convulsions in the legs. The mice died approximately 1 minute after injection. Therefore higher doses than 1000 nmol/kg were not tested. No statistical significant difference in the gridshock response was observed.

B2. Locomoter Test

The locomotor activity test was used to investigate horizontal locomotion in mice moving freely in a cage. See eg., Dauge, V., et al. Neuropsychophammacology, 25 (2001) 690; Florin, S., et al. Eur. J. Pharmacol., 317 (1996) 9; Jenck, F., et al. Proc. Natl. Acad. Sci. U.S.A, 94 (1997) 14854 and references cited therein.

The locomotor activity system consisted of a plexiglas cage (L 42 cm, W 23 cm, H 18 cm). The cage was equipped with 8 horizontal photocell detectors placed 3 cm above the cage floor and with a distance of 5 cm in between. These photocells measure horizontal activity. Each time the mouse moves and breaks a light beam, it was registered by photocells as one activity count. The activity counts were accumulated for each minute. The system was designed to distinguish between break counts and activity counts. Counting all the interruptions of the laser beams produced the break counts, whereas counting only the non-repeated interruptions of the laser beam produces the activity counts. That is, two repeated interruptions of the same laser beam were only registered as one activity count, but as two break counts. This design made it possible to distinguish between repeated movements e.g. scratching, and real movements of the mice.

After injection of test compound or vehicle the mice were placed individually in the activity cage. The activity was measured for 60 minutes with the detection starting 1 minute after the placement of the mouse. n=8 in both vehicle and test group. The mouse was placed in the activity cage directly after injection. As a decrease in locomotor activity was expected to be measured, the mice were tested using a foreign cage as test cage. No persons other then the investigator were allowed in the room, and the test was performed in a sound attenuated room. On the day of testing the mice should not be moved from their home cage, before the test is initiated. Before the eight test compounds were tested, the locomotor activity test was validated with chlorpromazine, amphetamine, and nociceptin.

B2. Data Analysis

The data obtained in the locomotor activity test was treated statistically by a one-way analysis of variance (ANOVA) and a post-hoc test. The post-hoc test used was The Fisher Least Significant difference (LSD) method. The statistical analyses were performed on the basis of AUC, calculated from the time-response curve. AUC₀₋₃₀ₘᵢₙ made the basis for the. The time denotation refers to the time after injection of compound. It was tested statistically, if all the vehicle groups tested in relation to one test compound are significant different from each other. When no statistical significance was found, a pooled group containing all the vehicle groups related to the compound in question, was used in the statistical analysis of the test compound. If the vehicle groups connected to one test compound were significant different, no pooling of the vehicle groups were performed, and the statistical analysis of one dose was made comparing the dose with the vehicle group run together with the dose in question.

B3. Locomotor Test Results

In the locomotor activity test the accumulated activity counts per minute are presented in a time-response curve with the activity count as a function of the time after injection.

Nociceptin was tested i.v. in the doses 30, 3000 and 10000 nmol/kg. A statistical significant increase in locomotor activity was observed at the dose 10000 nmol/kg during the first 30 minutes (p<0.05). Nociceptin was tested i.c.v. in the doses 0.003, 0.3 and 1 nmol/mouse. A Statistical significant decrease in locomotor activity was observed from 15-30 minutes at the dose 1 nmol/mouse (p<0.05).

TFA was tested in a dose of 50000 nmol/kg i.v. No statistical significant difference in the activity count was observed during the first 30 minutes.

Compound 40 was tested i.v. in the doses 3, 300, 1000 and 2000 mmol/kg. A Statistical significant decrease in locomotor activity was observed from 15-30 minutes at the doses 1000 and 2000 nmol/kg. A Statistical significant increase in locomotor activity was observed during the last 30 minutes at the doses 1000 nmol/kg (p<0.05).

Compound 40 in the dose 1000 nmol/kg i.v. was also tested in home cages. This was due to the terminal increase in locomotor activity observed under normal test conditions in foreign cages. When using home cages an increase in locomotor activity is better detected. A Statistical significant decrease in locomotor activity was observed during the first 30 minutes, and a statistical significant increase was observed from 31-59 minutes when using home cages (p<0.05).

Compound 11 was tested i.v. in the doses 100 and 10000 nmol/kg. No statistical significant difference in the locomotor activity was observed during the first 30 minutes.

Compound 28 was tested i.v. in the doses 30, 3000 and 10000 nmol/kg. A statistical significant decrease in locomotor activity was observed at the dose 10000 nmol/kg during the first 30 minutes p<0.05).

Compound 29 was tested i.v. in the doses 3, 100, 300 and 1000 nmol/kg. A statistical significant decrease in locomotor activity was observed at the doses 300 and 1000 mmol/kg during the first 30 minutes (p<0.05).

Compound 35 was tested i.v. in the doses 3, 300 and 1000 mmol/kg. A statistical significant decrease in locomotor activity was observed at the dose 1000 nmol/kg during the first 30 minutes (p<0.05).

Compound 39 was tested i.v. in the doses 3, 300, 1000, 3000 and 5000 nmol/kg. A statistical significant decrease in locomotor activity was observed at the doses 1000, 3000 and 5000 nmol/kg during the first 30 minutes (p<0.05).

Compound 34 was tested i.v. in the doses 3, 300 and 1000 nmol/kg. No statistical significant difference in the activity count was observed during the first 30 minutes, but a statistical significant decrease in locomotor activity was observed at the dose 1000 nmol/kg during the first 15 minutes (p<0.05).

Compound 36 was tested i.v. in the doses 1, 100, 300 and 1000 nmol/kg. A statistical significant decrease in locomotor activity was observed at the doses 300 and 1000 nmol/kg during the first 30 minutes (p<0.05).

C. Data Summary

The effect seen after i.v. administration of Compounds 40, 28, 29, 35 and 39 was nearly the same effect as observed after i.t. injection of nociceptin. See King, M. A., et al. Neurosci. Lett., 223 (1997) 113. The observed effect was analgesia.

Results of the gridshock and locomoter tests are shown below in Table 6 for eight 3-substituted 1,3,8-triaza-spiro[4.5]decan-4-ones. Also shown is oral bioavailability (F %) and half-life (T_{1/2} min) as determined by methods described previously.

TABLE 6

| | Cmp | Cmp | Cmp | Cmp | Cmp | Cmp | Cmp | Cmp |
|---|---|---|---|---|---|---|---|---|
| | 40 | 11 | 28 | 29 | 35 | 39 | 34 | 36 |
| F % | 4 ± 0.3 | 11 ± 3 | 42 ± 15 | <1 | <1 | <1 | | IC |
| T½ min | 36 | 25 | 420 | 45 | 225 | 270 | | 120 |
| CNS grid | 300 | >10000 | 3000 | <100 | 100 | 3000 | 300 | 1000 |
| shock* | | | | | | | | |
| CNS motility* | 300 | >10000 | 3000 | <300 | 300 | <1000 | 100 | 100 |

IC: Inconclusive: very large differences between the individual animals
*Minimal effective dose nmol/kg

### DISCUSSION

The foregoing examples show that some of the polar tailed 1,3,8-triaza-spiro[4.5]decan-4-ones are effective when given orally. Certain compounds containing many amines like Compounds 29, 30 and 39 are orally available and do significantly increase diuresis. Further, the acid Compound 11 is orally available. Compound 11 and 28 have an orally bioavailability of more than 1%.

The examples also show only small differences in hORL1 receptor binding as a function of polar tail length. C₆ and C₉ polar tails were generally a little better than C₃. This trend was not dependent on the amine part, but the difference is dependent on the amine-entity.

Varying the amine entity shows that more amino groups in the amine entity increases the ORL1 receptor binding as well as the efficacy. All compounds (Compounds 12, 14, 16, 31, 32, 26, 22, 29, 30, 27, 35, and 29) tested in efficacy assay show full agonism.

It will be appreciated that reference herein to “cmp” including plural forms means “compound” or “compounds”.

All references disclosed herein are incorporated by reference.

This invention has been described in detail with reference to preferred embodiments thereof. However, it will be appreciated that those skilled in the art, upon consideration of this disclosure, may make modifications and improvements within the spirit and scope of the invention.

1. A compound represented by the following formula I: 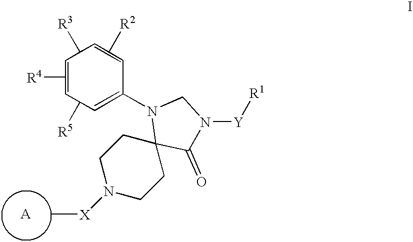
wherein,
(a) Y is 0, an optionally substituted C₁₋₁₂ alkylene, C₁₋₁₂ alkenylene, C₁₋₁₂ alkynylene group, 2-6 peptidyl residue, or poly oxyalkyl or combinations thereof in which each alkyl, alkenyl or alkynyl group is branched or unbranched,
(b) R¹ is —NR⁶, R⁷, R⁸ in which each of R⁶ and R⁷ is independently H or optionally substituted lower alkyl or R⁶ is —(CH₂)ₙ₁—NHR₇ in which n1 is between from about 1 to about 20 and R⁸ is 0, H or optionally substituted lower alkyl,
(c) R¹ is —NR³—[(CH₂)ₙ₂—NH]ₙ₃—(CH₂)ₙ₄—R⁹ in which n2 and n3 are each independently 1 to about 10, n4 is 1 to about 6, R⁹ is —NR⁶, R⁷, cyano, or an optionally substituted hydrazine, guanidine, azole or azine group,
(d) R¹ is —NH—[(CH₂)ₙ₁—NH]ₙ₂—(CH₂)ₙ₃—X2 in which each of R¹⁰ and R¹¹ is independently —NR⁶, R⁷, —CH═NH, cyano, or 0, provided that both of R¹⁰ and R¹¹ are not 0, wherein X2 is represented by the following formula 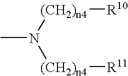
or
(e) R¹ is represented by the following group: 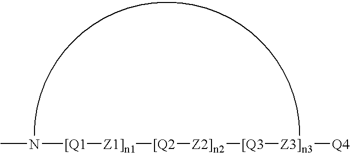
in which each of Q1, Q2, Q3 and Q4 are independently an optionally substituted lower alkyl, lower oxyalkyl, α,ω-dioxo-lower alkyl, or aryl alkyl group, and each of Z1, Z2 and Z3 is independently N, O or S,
(f) R¹ is an optionally substituted lower alkoxy, lower alkylcarboxy group, allyl, halogen, benzoxy, or a Boc protecting group,
(g) A is an optionally substituted C₅₋₁₂ cyclohexyl, phenyl, aminophenyl, cyanophenyl, cyanodiphenylmethyl, phenoxy, benzodioxinyl, cyanodiphenylmethyl, napthyl, anthryl, furanyl, indanyl, azulenyl, indolyl, isoindolyl, benzothienyl, benzofuranyl, bicyclo[6.2.0]dec-9-yl, acenapthenyl, bicyclo[3.3.1]non-9-yl, phenalenyl, indenyl, bicyclo[3.1.0]hex-3-yl, or coumarinyl group,
(h) X is 0, or an optionally substituted lower alkyl, lower alkenyl, or lower alkynyl group provided that when R¹ is an amino or guanidino and A comprises at least one aromatic group as defined above, then X is 0;
(i) R², R³, R⁴, and R⁵ are each independently H, halogen or an optionally substituted lower alkyl, provided that when A comprises a phenyl group annulated or as a substituent, then R¹ comprises more than one amino or guanidino group;
and a salt or solvate thereof.

2. The compound of claim 1, wherein the compound is represented by the following formula II: 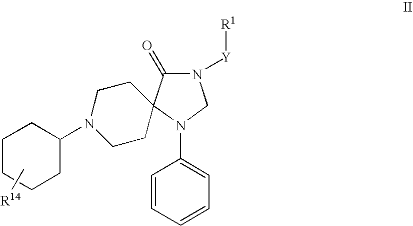
wherein R¹⁴ is halogen, cyano, hydroxy, nitro, or an optionally substituted lower alkyl, lower alkenyl, lower alkynyl or lower alkoxy group.

3. The compound of claim 2, wherein R¹² is a lower alkyl group.

4. The compound of claim 3, wherein the lower alkyl is n-propyl or isopropyl.

5. The compound of claim 3, wherein the lower alkyl group is bound to the 4-position of the cyclohexyl ring.

6. The compound of claim 1, wherein R¹ comprises a primary amine group or a polyamine.

7. The compound of claim 1, wherein R¹ comprises a secondary amine group.

8. The compound of claim 1, wherein R¹ comprises a tertiary amine group.

9. The compound of claim 1, wherein R¹ comprises a cyclic amine group.

10. The compound of claim 2, wherein the lower alkyl group is cis to the nitrogen atom of the azine ring.

11. The compound of claim 1, wherein R¹ has a molecular weight of less than about 1000 Da.

12. The compound of claim 1, wherein the R¹ group has a net positive charge of between 1 to about 10 at a pH of about 7.5.

13. The compound of claim 1, wherein the compound has an oral bioavailability (F %) of at least about 5% as determined by the standard plasma test.

14. The compound of claim 1, wherein the compound has an oral bioavailability (F %) of between from about 20% to about 75% as determined by the standard plasma test.

15. The compound of claim 1, wherein the compound exhibits an increase in diuresis of at least 1.2 as determined by a standard diuresis test.

16. The compound of claim 15, wherein the increase in diuresis is between 1.5 to about 5.0 as determined in the standard diuresis test.

17. The compound of claim 1, wherein the compound exhibits an IC₅₀ of at least about 10.1M in a standard hORL-1 receptor binding assay.

18. The compound of claim 17, wherein the compound exhibits an IC₅₀ of between from about 5 nM to about 100 nM in the standard hORL-1 receptor binding assay.

19. The compound of claim 1, wherein the compound exhibits an EC₅₀ of less than about 50 nM in a standard forskoline-induced cAMP assay.

20. The compound of claim 2 selected from one of the following:
(a) cis-3-(6-Methylamino-hexyl)-8-(4-isopropyl-cyclohexyl)-1-phenyl-1,3,8-triaza-spiro[4.5]decan-4-one (Compound 12),
(b) trans-3-(6-Methylamino-hexyl-8-(4-isopropyl-cyclohexyl)-1-phenyl-1,3,8-triaza-spiro[4.5]decan-4-one (Compound 13),
(c) cis-3-N-(6-Methylaminohexyl)-(6-methylaminohexyl)-8-(4-isopropyl cyclohexyl)-1-phenyl-1,3,8-triaza-spiro[4.5]decan-4-one (Compound 14),
(d) trans-3-N-(6-Methylaminohexyl)-(6-methylaminohexyl)-8-(4-isopropyl-25 cyclohexyl)-1-phenyl-1,3,8-triaza-spiro[4.5]decan-4-one (Compound 15),
(e) cis-3-(3-Amino-propyl)-8-(4-isopropyl-cyclohexyl)-4-phenyl-1,3,8-triaza-spiro[4.5]decan-4-one (Compound 16),
(f) trans-3-(3-Amino-propyl)-8-(4-isopropyl-cyclohexyl)-1-phenyl-1,3,8-triaza-spiro[4.5]decan-4-one (Compound 21),
(g) cis-3-(9-Amino-nonyl)-8-(4-isopropyl-cyclohexyl)-1-phenyl1,3,8-triaza-spiro[4.5]decan-4-one (Compound 23),
(h) trans-3-(9-Amino-nonyl)-8-(4-isopropyl-cyclohexyl)-1-phenyl-1,3,8-triaza-spiro[4.5]decan-4-one (Compound 29),
(i) cis-3-(13-Aminoethyl-10,13,16-triazahexadecan)-8-(4-isopropyl-cyclohexyl)-1-phenyl-1,3,8-triaza-spiro[4.5]decan-4-one (Compound 31),
(j) trans-3-(13-Aminoethyl-10,13,16-triazahexadecan)-8-(4-isopropyl-cyclohexyl)-1-phenyl-1,3,8-triaza-spiro[4.5]decan-4-one (Compound 32),
(k) cis-3-(3-Dimethylamino-propyl)-8-(4-isopropyl-cyclohexyl)-1-phenyl-1,3,8-triaza-spiro[4.5]decan-4-one (Compound 26),
(l) cis-3-(6-Amino-hexyl)-8-(4-isopropyl-cyclohexyl)-1-phenyl-1,3,8-triaza-spiro[4.5]decan-4-one (Compound 22),
(m) cis-3(9-Dimethylamino-nonyl)-8-(4-isopropyl-cyclohexyl)-1-phenyl-1,3,8-triaza-spiro[4.5]decan-4-one (Compound 28),
(n) cis-3-(7-Aminoethyl-4,7,10-triazadecan)-8-(4-isopropyl-cyclohexyl)-1-phenyl-1,3,8-triaza-spiro[4.5]decan-4-one (Compound 29),
(o) cis-3-(10-Aminoethyl-7,10,13-triazamidecan)-8-(4-isopropyl-cyclohexyl)-1-phenyl-1,3,8-triaza-spiro[4.5]decan-4-one (Compound 30),
(p) cis-3-(6-Dimethylamino-hexyl)-8-(4-isopropyl-cyclohexyl)-1-phenyl-1,3,8-triaza-spiro[4.5]decan-4-one (Compound 27),
(q) cis-8-(4-Isopropyl-cyclohexyl)-3-(10,14,17,20,23-pentaazatricosanyl) 1-phenyl-1,3,8-triaza-spiro[4.5]decan-4-one (Compound 35),
(r) cis-8-(4-Isopropyl-cyclohexyl)-1-phenyl-3-[9-(1,4,8,11-tetraaza-cyclotetradec-1-yl)-nonyl]-1,3,8-triaza-spiro[4.5]decan-4-one (Compound 39),
(s) cis-8-(4-Isopropyl-cyclohexyl)-3-(7,10,14,17,20-pentaazaeicosanyl)-1-phenyl-1,3,8-triaza-spiro[4.5]decan-4-one (Compound 34),
(t) cis-8-(4-Isopropyl-cyclohexyl)-3-(4,7,10,14,17-pentaazaheptadecyl)-1-phenyl-1,3,8-triaza-spiro[4.5]decan-4-one (Compound 33),
(u) cis-8-(4-Isopropyl-cyclohexyl)-1-phenyl-3-[3-(1,4,8,11-tetraaza-cyclotetradec-1-yl)-propyl]-1,3,8-triaza-spiro[4.5]decan-4-one (Compound 36); and
(v) cis-8-(4-Isopropyl-cyclohexyl)-1-phenyl-3-[6-(1,4,8,11-tetraaza-cyclotetradec-1-yl)-hexyl]-1,3,8-triaza-spiro[4.5]decan-4-one (Compound 38)
and a salt or a solvate thereof, preferably a pharmaceutically acceptable salt.

21. A compound represented by the following formula III: 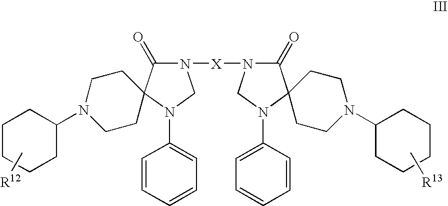
wherein,
(a) R¹² and R¹³ are each independently an optionally substituted lower alkyl or lower alkoxy group,
(b) X is an optionally substituted lower alkyl group, polyethylene glycol (PEG), polyamine, disulfide, or a 2-6 peptidyl residue;
and a salt or solvate thereof.

22. The compound of claim 21, wherein the lower alkyl or lower alkoxy group is substituted with at least one of halogen, cyano, hydroxy or nitro.

23. The compound of claim 21, wherein X is a lower alkyl group substituted with between from 1 to about 5 nitrogen atoms.

24. The compound of claim 21, wherein R² and R³ are each independently an unsubstituted lower alkyl group the same or different.

25. The compound of claim 24, wherein R² and R³ are each n-propyl or isopropyl.

26. The compound of claim 25, wherein the n-propyl or isopropyl group is linked to the cyclohexyl group at the 4-position.

27. The compound of claim 21, wherein X is pentyl, hexyl, heptyl, octyl, nonyl, or decyl group.

28. The compound of claim 21, wherein X is 5-azaundecan, 6-azamidecan, 7-azapentadecanl, 8-azaheptadecan, 9-aza-nonadecan or 10-azaundodecan.

29. The compound of claim 21, wherein X is an 5-azaundecan-1,1′-diyl, 6-azamidecan 1,13-diyl, 7-azapentadecanl-1,15-diyl, 8-azaheptadecan 1,17-diyl, 9-aza-nonadecan-1,1,9-diyl or a 10-azaundodecan-1,2′-diyl group.

30. The compound of claim 21, wherein the compound exhibits an increase in diuresis of at least 1.2 as determined by a standard diuresis test.

31. The compound of claim 30, wherein the increase in diuresis is between 1.5 to about 5.0 as determined in the standard diuresis test.

32. The compound of claim 21, wherein the compound exhibits an IC₅₀ of at least about 0.1 nM in a standard hORL-1 receptor binding assay.

33. The compound of claim 32, wherein the compound exhibits an IC₅₀ of between from about 5 nM to about 100 nM in the standard hORL-1 receptor binding assay.

34. The compound of claim 21, wherein the compound exhibits an EC₅₀ of less than about 50 nM in a standard forskoline-induced cAMP assay.

35. The compound of claim 21, wherein the compound is one of the following:
(i) bis-(cis-3-Propyl-8-(4-isopropyl-cyclohexyl)-1-phenyl-1,3,8-triaza-spiro[4.5]decan-4-on)-amine (Compound 19),
(ii) bis-(trans-3-Propyl-8-(4-isopropyl-cyclohexyl)-1-phenyl-1,3,8-triaza-spiro[4.5]decan-4-on)-amine (Compound 20), or
(iii) 1,9-bis-(cis-1-8-(4-isopropyl-cyclohexyl)-1-phenyl-1,3,8-triaza-spiro[4.5]decan-4-one-3-yl)-nonane (Compound 25).

36. A composition comprising at least one of the compounds of claim 1 or claim 21 and at least one pharmaceutically acceptable carrier or vehicle.

37. A method of making the compound of claim 1, the method comprising at least one of the following steps:
a) alkylating the 3-position of a 1-phenyl-1,3,8-triaza-spiro[4.5]decan-4-one,
b) aminating the product of step a) under reducing conditions sufficient to add the A ring to the 8-position of the product,
c) brominating the alkyl group added to the 3-position of the product of Step b) to produce a bromide; and
d) substituting the bromine with the R¹ group to make the compound.

38. The method of claim 37, wherein prior to step a), the 1-phenyl-1,3,8-triaza-spiro[4.5]decan-4-one is protected in the 8-position.

39. The method of claim 38, wherein after step a), the 8-position of the product is deprotected.

40. The method of claim 37, wherein the method further comprises the step of separating the compounds into cis and trans isomers.

41. The method of claim 40, wherein the separation step is conducted prior to step d).

42. The method of claim 41, wherein the separation step is conducted between steps c) and d).

43. A method of modulating diuresis in a mammal, the method comprising administering the composition of claim 36 in an amount sufficient to modulate the diuresis in the mammal.

44. A method of modulating aquaresis in a mammal, the method comprising administering the composition of claim 36 in an amount sufficient to modulate the aquaresis in the mammal.

45. A method for preventing or treating edema in a mammal, the method comprising administering the composition of claim 36 in an amount sufficient to prevent or treat the edema.

46. The method of claim 45, wherein the edema is pulmonary edema or edema associated with hyponatremia.

47. A method of modulating arterial blood pressure in a mammal, the method comprising administering the composition of claim 36 in an amount sufficient to modulate the arterial blood pressure in the mammal.

48. A method of antagonizing the nociceptin (ORL1) receptor, the method comprising contacting the receptor with an effective amount of at least one of the compounds of claim 1 or claim 21.

49. A method of sedating a mammal, the method comprising administering to the mammal a therapeutically effective amount of at least one of the compounds of claim 36.

50. The method of claim 49, wherein the compound comprises at least one of the following: a) cis-3(9-Dimethylamino-nonyl)-8-(4-isopropyl-cyclohexyl)-1-phenyl-1,3,8-triaza-spiro[4.5]decan-4-one (Compound 28); b) cis-3-(7-aminoethyl-4,7,10-triazadecan)-8-(4-isopropyl-cyclohexyl)-1-phenyl-1,3,8-triaza-spiro[4.5]decan-4-one (Compound 29); c) cis-9-(Tetraethylenpentamin)-nonyl-8-(4-isopropyl-cyclohexyl)-1-phenyl-1,3,8-triaza-spiro[4.5]decan-4-one (Compound 35); and d) cis-9-(1,4,8,11-tetraazacyclotetradecane)-nonyl-8-(4-isopropyl-cyclohexyl)-1-phenyl-1,3,8-triaza-spiro[4.5]decan-4-one (Compound 39).

51. A method for reducing nociception in a mammal, the method comprising administering a therapeutically effective amount of at least one of the compounds of claim 36.

52. The method of claim 51, wherein the compound comprises at least one of the following: a) cis-3(9-Dimethylamino-nonyl)-8-(4-isopropyl-cyclohexyl)-1-phenyl-1,3,8-triaza-spiro[4.5]decan-4-one (Compound 28); b) cis-3-(7-aminoethyl-4,7,10-triazadecan)-8-(4-isopropyl-cyclohexyl)-1-phenyl-1,3,8-triaza-spiro[4.5]decan-4-one (Compound 29); c) cis-9-(Tetraethylenpentamin)-nonyl-8-(4-isopropyl-cyclohexyl)-1-phenyl-1,3,8-triaza-spiro[4.5]decan-4-one (Compound 35); d) cis-9-(1,4,8,1,1-tetraazacyclotetradecane)-nonyl-8-(4-isopropyl-cyclohexyl)-1-phenyl-1,3,8-triaza-spiro[4.5]decan-4-one (Compound 39); and e) cis-3-(1,4,8,11-tetraazacyclotetradecane)-propyl-8-(4-isopropyl-cyclohexyl)-1-phenyl-1,3,8-triaza-spiro[4.5]decan-4-one, (Compound 36).
